SEQUENCE LISTING (22 sequences)

SEQ ID NO: 1 - protein, 455 aa, Artificial Sequence
  Description of Artificial Sequence Synthetic polypeptide
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Artificial Sequence
  Description of Artificial Sequence Synthetic polypeptide
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 455 aa, Artificial Sequence
  Description of Artificial Sequence Synthetic polypeptide
  MOD_RES (412)..(412): Non natural amino acid
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 4 - protein, 455 aa, Artificial Sequence
  Description of Artificial Sequence Synthetic polypeptide
  MOD_RES (188)..(188): Non natural amino acid
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 215 aa, Artificial Sequence
  Description of Artificial Sequence Synthetic polypeptide
  MOD_RES (43)..(43): Non natural amino acid
MDIQMTQSPSSLSASVGDRVTITCRASQDVNTAVAWYQQKPGXAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 455 aa, Artificial Sequence
  Description of Artificial Sequence Synthetic polypeptide
  MOD_RES (188)..(188): Non natural amino acid
  MOD_RES (412)..(412): Non natural amino acid
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 7 - protein, 455 aa, Artificial Sequence
  Description of Artificial Sequence Synthetic polypeptide
  MOD_RES (249)..(249): Non natural amino acid
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVXLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 455 aa, Artificial Sequence
  Description of Artificial Sequence Synthetic polypeptide
  MOD_RES (188)..(188): Non natural amino acid
  MOD_RES (412)..(412): Non natural amino acid
MEVQLVESGGGLVQPGGSLRLSCAASGFNIRTQSIHWVRQAPGKGLEWIGDIFPIDGITDYADSVKGRFTISADTSKNTAYLQMNSLRAEDTAVYYCARGSWSWPSGMDYYLDYWGQGTLVTVSSASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 9 - protein, 215 aa, Artificial Sequence
  Description of Artificial Sequence Synthetic polypeptide
MDIQMTQSPSSVSASVGDRVTITCRASQGIGSWLAWYQQKPGKAPKLLIYAADRLQSGVPSRFSGSGSGTDFTLTISSLQPEDFATYYCQQYHTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 454 aa, Artificial Sequence
  Description of Artificial Sequence Synthetic polypeptide
  MOD_RES (411)..(411): Non natural amino acid
MQVQLVESGGGVVQPGRSLRLSCAASGFNFSDYGMHWVRQAPGKGLEWVAVIWYDGSISYYADSVKGRFTISRDNSKNTLYLQMNSLRAEDTAVYYCARGGTVEHGAVYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 11 - protein, 450 aa, Artificial Sequence
  Description of Artificial Sequence Synthetic polypeptide
MEVQLVQSGAEVKKPGASVKVSCKASGYTFDSYGISWVRQAPGQGLEWMGWISAYNGNTNYAQKLQGRVTMTTDTSTNTAYMELRSLRSDDTAVYYCARDVDYGTGSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial Sequence
  Description of Artificial Sequence Synthetic polypeptide
MSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 452 aa, Artificial Sequence
  Description of Artificial Sequence Synthetic polypeptide
MEVQLVESGGGLVQPGGSLRLSCAASGFNIDRYYIHWVRQAPGKGLEWVAGITPVRGYTEYADSVKDRFTISADTSKNTAYLQMNSLRAEDTAVYYCARGYVYRMWDSYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 215 aa, Artificial Sequence; identical to SEQ ID NO: 2
  Description of Artificial Sequence Synthetic polypeptide

SEQ ID NO: 15 - protein, 453 aa, Artificial Sequence
  Description of Artificial Sequence Synthetic polypeptide
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 216 aa, Artificial Sequence
  Description of Artificial Sequence Synthetic polypeptide
MEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 455 aa, Artificial Sequence
  Description of Artificial Sequence Synthetic polypeptide
MEVQLVESGGGLVQPGGSLRLSCAASGFNIRTQSIHWVRQAPGKGLEWIGDIFPIDGITDYADSVKGRFTISADTSKNTAYLQMNSLRAEDTAVYYCARGSWSWPSGMDYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 454 aa, Artificial Sequence
  Description of Artificial Sequence Synthetic polypeptide
MQVQLVESGGGVVQPGRSLRLSCAASGFNFSDYGMHWVRQAPGKGLEWVAVIWYDGSISYYADSVKGRFTISRDNSKNTLYLQMNSLRAEDTAVYYCARGGTVEHGAVYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 20 bp, Artificial Sequence
  Description of Artificial Sequence Synthetic oligonucleotide
taatacgactcactataggg

SEQ ID NO: 20 - protein, 451 aa, Artificial Sequence
  Description of Artificial Sequence Synthetic polypeptide
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 7 bp, Artificial Sequence
  Description of Artificial Sequence Synthetic oligonucleotide
argagrt

SEQ ID NO: 22 - DNA, 7 bp, Artificial Sequence
  Description of Artificial Sequence Synthetic oligonucleotide
arrakat

(19) United States
(12) Patent Application Publication
(10) Pub. No.: US 2022/0089690 A1
(43) Pub. Date: Mar. 24, 2022

(54) METHOD FOR LARGE SCALE PRODUCTION OF ANTIBODIES USING A CELL-FREE PROTEIN SYNTHESIS SYSTEM

(71) Applicant: Sutro Biopharma, Inc., South San Francisco, CA (US)

(72) Inventor: Gang Yin, South San Francisco, CA (US)

(21) Appl. No.: 17/473,544

(22) Filed: Sep. 13, 2021

Related U.S. Application Data
(60) Provisional application No. 63/078,254, filed on Sep. 14, 2020.

(51) Int. Cl. C07K 16/06; C07K 16/28

(52) U.S. Cl. CPC C07K 16/06; C07K 16/2878; C07K 16/28; C07K 2317/622; C07K 2317/55; C07K 2317/31; C07K 2317/526; C07K 16/2863

(57) ABSTRACT

Described herein are methods for large scale production of antibodies using a cell-free protein synthesis system. The methods include expressing a heavy chain (HC) polypeptide of an antibody from a nucleic acid encoding the heavy chain in a cell-free bacterial extract in the presence of a light chain (LC) polypeptide, thereby producing the antibody. The methods are performed at a large scale that is suitable for commercial production of antibodies, for example in a reaction volume equal to or greater than about 10 liters, for example about 10 to about 25,000 liters. The methods result in increased yields per unit volume of properly folded and assembled antibodies as opposed to synthesizing the light chain in the same cell-free protein synthesis system as the heavy chain polypeptide.

### CROSS-REFERENCES TO RELATED APPLICATIONS

This application is a U.S. Non-Provisional which claims the benefit of priority to U.S. Provisional Application No. 63/078,254, filed on Sep. 14, 2020, the disclosure of which is hereby incorporated by reference in its entirety herein for all purposes.

### SEQUENCE LISTING

The instant application contains a Sequence Listing which has been submitted electronically in ASCII format and is hereby incorporated by reference in its entirety. Said ASCII copy, created on Sep. 7, 2021, is named 091200-1261027-006910US SL.txt and is 65,782 bytes in size.

### BACKGROUND

The present disclosure provides methods and systems for producing proteins of interest at large scale suitable for commercial production. The methods can increase the total yield and/or the yield of properly folded and assembled proteins of interest, such as antibodies.

### BRIEF SUMMARY OF THE INVENTION

Described herein are methods and compositions for increasing the yield of properly folded antibodies in a cell-free protein synthesis system. In one aspect, the methods are performed at a scale suitable for commercial production of relatively large amounts of properly folded antibodies. Thus, described herein is a method for large scale production of an antibody using a cell-free protein synthesis system, the method comprising expressing a heavy chain (HC) polypeptide of an antibody from a nucleic acid encoding the heavy chain in the presence of a light chain (LC) polypeptide, thereby producing the antibody. In some embodiments, the cell-free protein synthesis system comprises a reaction mixture having a volume of about 10 to about 25,000 liters. In some embodiments, expression of the HC is performed in a reaction mixture having a volume of about 10 to about 10,000 liters. In some embodiments, expression of the HC is performed in a reaction mixture having a volume of about 15,000 to about 25,000 liters. In some embodiments, the reaction mixture comprises a volume equal to or greater than about 10 liters to about 25,000 liters. In some embodiments, the reaction mixture comprises a volume equal to or less than about 10 liters to about 25,000 liters. In some embodiments, the reaction mixture comprises a volume of about 10,000 liters to about 20,000 liters.

In some embodiments, the reaction mixture comprises a bacterial extract, and expression of the HC comprises: (i) combining the bacterial extract with a nucleic acid encoding the HC; and (ii) incubating the cell-free protein synthesis system under conditions permitting the expression of the HC.

In some embodiments, the yield per liter of total antibody protein is increased compared to a reaction mixture where both the heavy and light chain polypeptides are expressed in the same reaction mixture. In some embodiments, the yield per liter of properly folded antibody protein(s) is increased compared to a reaction mixture where both the heavy and light chain polypeptides are expressed in the same reaction mixture. In some embodiments, the yield per liter of total antibody protein and the yield per liter of properly folded antibody protein(s) is increased compared to a reaction mixture where both the heavy and light chain polypeptides are expressed in the same reaction mixture. In some embodiments, the yield per liter of total antibody protein and/or the yield per liter of properly folded antibody protein(s) is increased compared to a reaction mixture where both the heavy and light chain polypeptides are expressed contemporaneously in the same reaction mixture.

In some embodiments, the yield per liter of properly folded antibody protein(s) is about 30% to about 90% higher or greater compared to a reaction mixture where both the heavy and light chain polypeptides are expressed. In some embodiments, the yield per liter of properly folded antibody protein(s) is about 30%, about 40%, about 50%, about 60%, about 70%, about 80% or about 90% higher or greater compared to a reaction mixture where both the heavy and light chain polypeptides are expressed. In some embodiments, dimerization between the heavy and light chain polypeptides is increased. In some embodiments, the ratio of heavy and light chain polypeptide dimers to heavy and light chain polypeptide monomers is increased.

In some embodiments, the light chain polypeptide is added to the reaction mixture prior to the expressing step.

In some embodiments, the light chain polypeptide is produced in a separate reaction before being added to the reaction mixture. In some embodiments, the light chain polypeptide is synthesized or prefabricated before being added to the reaction mixture. In some embodiments, the light chain polypeptide is expressed from a nucleic acid encoding the light chain polypeptide in a cell-free protein synthesis system, reaction mixture, or cell free extract. In some embodiments, the light chain polypeptide is expressed from a nucleic acid encoding the light chain polypeptide in an intact living cell. In some embodiments, the intact living cell is a bacterial cell or mammalian cell. In some embodiments, the light chain polypeptide is purified or partially purified from cell-free protein synthesis systems, reaction mixtures, cell free extracts, and/or from cultures of cells before being added to the reaction mixture.

In some embodiments, the antibody is an IgG, IgA, or IgD subtype, or a combination thereof. In some embodiments, the antibody is a monoclonal antibody. In some embodiments, the antibody is selected from an anti-B cell maturation antigen (anti-BCMA) antibody, an anti-Cluster of Differentiation 74 (anti-CD74) antibody, or an anti-folate receptor alpha (FOLR1) antibody. In some embodiments, the antibody comprises a FAB fragment.

In some aspects, the antibody is a bispecific antibody. In some embodiments, the bispecific antibody comprises a heterodimeric Fc region comprising two asymmetric CH3 domains that include sequences from IgA and IgG CH3 domains. In some embodiments, the bispecific antibody is a domain-exchanged antibody, wherein the HC dimerizes with the LC. In some embodiments, the bispecific antibody comprises engineered CH3 domains with enhanced HC heterodimerization based on steric or electrostatic complementarity. In some embodiments, the engineered CH3 domains comprise knob and hole mutations that promote the formation of stable CH3 heterodimers. In some embodiments, the bispecific antibody comprises one Fab domain and one scFv domain, where the Fab and scFv domains bind to different antigens.

In some aspects, the HC and/or the LC comprises at least one non-natural amino acid (nnAA). In some embodiments, the HC comprises at least one non-natural amino acid (nnAA). In some embodiments, the nnAA in the HC can be the same or different from the nnAA in the LC. In some embodiments, the nnAA is p-acetyl-phenylalanine or p-azidomethyl-L-phenylalanine.

In some embodiments, the method further comprises assembling the HC and LC under non-reducing conditions to produce the antibody.

In some embodiments, the cell free protein synthesis system comprises a bacterial extract with associated co-factors. In some embodiments, the cell free protein synthesis system comprises a bacterial extract prepared from an E. coli strain. In some embodiments, the cell free protein synthesis system comprises an oxidative phosphorylation reaction producing ATP. In some embodiments, the cell free protein synthesis system comprises a reconstituted ribosome system.

In some embodiments, the cell free protein synthesis system comprises an exogenous protein chaperone. In some embodiments, the exogenous protein chaperone is selected from the group consisting of a protein disulfide isomerase (PDI), a peptidyl prolyl cis-trans isomerase (PPI), or a deaggregase. In some embodiments, the PDI is selected from DsbA, DsbC or DsbG; the PPI is selected from FkpA, SlyD, tig, SurA, or Cpr6; and the deaggregase is selected from IbpA, IbpB, or Skp.

In some embodiments, the cell free protein synthesis system comprises a mutant Releasing Factor 1 protein (RF1).

Also provided is a cell-free protein synthesis system. In one aspect, the cell-free protein synthesis system comprises: (i) a reaction mixture comprising a bacterial cell extract; (ii) a nucleic acid encoding a heavy chain polypeptide; and (iii) a light chain polypeptide. In some embodiments, the cell-free protein synthesis system comprises a reaction mixture having a volume of about 10 to about 25,000 liters.

In some embodiments of the cell-free protein synthesis system, the light chain polypeptide is added to the reaction mixture. In some embodiments, the light chain polypeptide is not expressed in the reaction mixture, or the reaction mixture does not contain a plasmid encoding the light chain. In some embodiments, the light chain polypeptide is produced in a separate reaction, synthesized or prefabricated before being added to the reaction mixture. In some embodiments, the light chain polypeptide is expressed from a nucleic acid encoding the light chain polypeptide in a cell-free protein synthesis system, reaction mixture, or cell free extract. In some embodiments, the light chain polypeptide is expressed from a nucleic acid encoding the LC polypeptide in an intact living cell. In some embodiments, the intact living cell is a bacterial cell or mammalian cell. In some embodiments, the light chain polypeptide is purified or partially purified from cell-free protein synthesis systems, reaction mixtures, cell free extracts, or from cultures of cells before being added to the reaction mixture.

In some embodiments, the reaction mixture comprises ribosomes, ATP, amino acids, and tRNAs. In some embodiments, the bacterial cell extract is prepared from an E. coli strain.

In some embodiments, the cell-free protein synthesis system further comprises an exogenous protein chaperone. In some embodiments, the exogenous protein chaperone is selected from the group consisting of a protein disulfide isomerase (PDI), a peptidyl prolyl cis-trans isomerase (PPI), or a deaggregase. In some embodiments, the PDI is selected from DsbA, DsbC or DsbG; the PPI is selected from FkpA, SlyD, tig, SurA, or Cpr6; and the deaggregase is selected from IbpA, IbpB, or Skp.

In some embodiments, the cell-free protein synthesis system further comprises a mutant Releasing Factor 1 protein (RF1).

### BRIEF DESCRIPTION OF THE DRAWINGS

FIG. 1 shows anti-BCMA antibody titers in reactions containing an expression plasmid encoding the heavy chain (HC) polypeptide and a light chain (LC) polypeptide, where the LC was provided either as a prefabricated light chain (PFLC) polypeptide or expressed from an expression plasmid encoding the LC polypeptide. The reactions were performed in bioreactors or using the FlowerPlate® (FP) system (m2p-labs GmbH). The yield (titer) of an anti-BCMA antibody was increased when a PFLC polypeptide was added to the reaction as compared to adding an expression plasmid encoding the LC polypeptide.

FIG. 2 shows the anti-folate receptor alpha (FOLR1) antibody titer increased in a dose dependent manner with increasing PFLC concentration, and that the increase in titer was nearly doubled when using 1.0-1.2 g/L PFLC compared to the titer when the LC was expressed from a plasmid encoding the LC (0 g/L PFLC). The antibody titer was measured using PhyTip® Columns (PhyNexus, Inc.) or by HPLC.

FIG. 3 shows anti-FOLR1 antibody titers in bioreactors and in the FlowerPlate system where the LC was provided as a PFLC or expressed from a plasmid. The antibody titer was measured using PhyTip® Columns. The yield of anti-FOLR1 antibody was increased in the 0.2 L bioreactor when a PFLC polypeptide was added to the reaction as compared to adding an expression plasmid encoding the LC polypeptide.

FIG. 4 shows anti-FOLR1 antibody titers in bioreactors where the LC was provided as a PFLC or expressed from a plasmid. The antibody titer was measured using HPLC. The yield of anti-FOLR1 antibody was increased in the 0.2 L bioreactor when a PFLC polypeptide was added to the reaction as compared to adding an expression plasmid encoding the LC polypeptide.

FIG. 5 shows anti-FOLR1 antibody titration data using extracts comprising HC pDNA plasmids (3 mg/L and 6 mg/L) with two different concentrations of PFLC (0.5 g/L and 0.75 g/L). The control was 37.% percent of an extract comprising heavy chain and light chain expression plasmids (3 mg/L).

FIG. 6 shows anti-CD74 antibody titers in bioreactors and FP system reactions where the HC was expressed from a plasmid and the LC was provided as a PFLC or expressed from a plasmid. The anti-CD74 antibody titer increased about 80% in 0.2 L and 5 mL bioreactors when the LC was provided as a PFLC. The anti-CD74 antibody titer increase increased about 50% in the 1 mL FlowerPlate when the LC was provided as a PFLC. The data in FIG. 6 is from a batch XCF, without feeding in the reactor.

FIG. 7 shows extracts containing PFLC increased the titer of anti-CD74 antibody compared to control extracts containing plasmid DNA encoding the light chain.

FIG. 8 shows relative expression of Trastuzumab IgG comparing HC/LC co-expression from plasmids to expression in reactions containing purified PFLC reagent or crude PFLC lysate reagent. Reactions comprising either the purified or crude PFLC reagents showed a similar increase in titer compared to reactions in which the HC and LC are co-expressed from plasmids.

FIG. 9 shows that the yield of SEEDbody Fab/scFv bispecific antibody was increased in reactions containing the purified PFLC reagent as compared to a reaction containing the LC expressed from a plasmid. The HC-GA and scFv-AG proteins were expressed from plasmids in both reactions.

FIG. 10 shows the titer of anti-BCMA antibody increased in a dose-response relationship based on the concentration of HC expression plasmid and PFLC added to the reaction. The results are expressed as a contour figure created on JMP.

FIG. 11 shows the titer of anti-FOLR1 antibody increased in a dose-response relationship based on the concentration of HC expression plasmid and PFLC added to the reaction. The results are expressed as a contour figure created on JMP.

FIG. 12 shows the titer of anti-CD74 antibody in reactions containing an HC expression plasmid and a PFLC. The results are expressed as a contour figure created on JMP.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N Y 1989); Ausubel et al., CURRENT PROTOCOLS IN MOLECULAR BIOLOGY, John Wiley and Sons (Hoboken, N Y 1995). The term “a” or “an” is intended to mean “one or more.” The term “comprise” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

The term “about,” denotes a range of ±10% of a reference or numerical value, for example, plus or minus 1%, 2%, 3%, 4%, 5%, 6%, 7%, 8%, 9% or 10% of a reference or numerical value.

The term “antibody” refers to a protein functionally defined as a binding protein and structurally defined as comprising an amino acid sequence that is recognized by one of skill in the art as being derived from the framework region of an immunoglobulin encoding gene of an animal producing antibodies. The term includes a single chain polypeptide or a double-chained polypeptide dimer comprising at least one set or pair of a heavy chain variable domain and a light chain variable domain that form a functional antigen binding site with a predetermined antigen specificity. The term includes monoclonal antibodies, bispecific antibodies and bispecific antibodies comprising modified Fc regions that promote formation of heterodimers. The term also includes bispecific antibodies comprising a FAB that binds a first antigen and an scFv that binds a second antigen. An antibody can consist of one or more polypeptides substantially encoded by immunoglobulin genes or fragments of immunoglobulin genes. In humans, the recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins, conjugates thereof (e.g., chimeric or bispecific antibodies), antigen-binding fragments thereof (e.g., Fab, F(ab′)2, Fv, dsFv, Fd and Fd′ fragments, diantibodies or diabodies (dAb), miniantibodies) and other compositions such as single chain antibodies (antibodies that exist as a single polypeptide chain), single chain Fv antibodies (sFv or scFv) in which a variable heavy and a variable light chain are joined together (directly or through a peptide linker) to form a continuous fusion polypeptide, and scFv-Fc fusion proteins. The term “antibody fragment” refers to any portion of a full-length antibody that is less than full length but contains at least a portion of the variable region of the antibody sufficient to form an antigen binding site (e.g., one or more CDRs) and thus retains the a binding specificity and/or an activity of the full-length antibody. The fragment can include multiple chains linked together, such as by disulfide bridges and/or by peptide linkers. See, e.g., METHODS IN MOLECULAR BIOLOGY, Vol 207: Recombinant Antibodies for Cancer Therapy Methods and Protocols (2003); Chapter 1; pp. 3-25, Kipriyanov; and Fundamental Immunology, W. E. Paul, ed., Raven Press, N.Y. (1993), for a more detailed description of antibody fragments.

The term “heavy chain polypeptide” refers to an Ig polypeptide that comprises a full length heavy chain from an antibody, or a fragment thereof that is capable of forming an antigen biding site when paired with or dimerized with a light chain polypeptide. The term includes fragments comprising the heavy chain variable domain (V_{H}).

The term “light chain polypeptide” refers to an Ig polypeptide that comprises a full length light chain from an antibody, or a fragment thereof that is capable of forming an antigen biding site when paired with or dimerized with a heavy chain polypeptide. The term includes fragments comprising the light chain variable domain (V_{L}).

The term “pre-fabricated light chain” or “pre-fabricated light chain polypeptide” refers to a LC polypeptide that is made, produced or synthesized before being added to another cell-free protein synthesis system, reaction mixture, or cell free extract. The term includes LC polypeptides that are manufactured or produced using any method known in the art. The term also includes LC polypeptides that are expressed from a nucleic acid encoding the LC polypeptide in a cell-free protein synthesis system, reaction mixture, or cell free extract. The term also includes LC polypeptides that are expressed from a nucleic acid encoding the LC polypeptide in an intact living cell, such as a bacterial cell or mammalian cell. The term also includes LC polypeptides that are purified or partially purified from cell-free protein synthesis systems, reaction mixtures, or cell free extracts, or from cultures of cells.

The term “bacterial derived cell free extract” refers to preparation of in vitro reaction mixtures able to transcribe DNA into mRNA and/or translate mRNA into polypeptides. The mixtures include ribosomes, ATP, amino acids, and tRNAs. They may be derived directly from lysed bacteria, from purified components or combinations of both.

The term “bacterial cell free synthesis system” refers to the in vitro synthesis of polypeptides in a reaction mix comprising biological extracts and/or defined reagents. The reaction mix will comprise a template for production of the macromolecule, e.g. DNA, mRNA, etc.; monomers for the macromolecule to be synthesized, e.g. amino acids, nucleotides, etc.; and co-factors, enzymes and other reagents that are necessary for the synthesis, e.g. ribosomes, uncharged tRNAs, tRNAs charged with unnatural amino acids, polymerases, transcriptional factors, tRNA synthetases, etc.

The term “peptide,” “protein,” and “polypeptide” are used herein interchangeably and refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins and truncated proteins, wherein the amino acid residues are linked by covalent peptide bonds.

As used herein, the term “Fab fragment” is an antibody fragment that contains the portion of the full-length antibody that results from digestion of a full-length immunoglobulin with papain, or a fragment having the same structure that is produced synthetically, e.g. recombinantly. A Fab fragment contains a light chain (containing a variable (V_{L}) and constant (C_{L}) region domain) and another chain containing a variable domain of a heavy chain (V_{H}) and one constant region domain portion of the heavy chain (C_{H1}).

As used herein, a F(ab′)₂ fragment is an antibody fragment that results from digestion of an immunoglobulin with pepsin at pH 4.0-4.5, or a synthetically, e.g. recombinantly, produced antibody having the same structure. The F(ab′)₂ fragment contains two Fab fragments but where each heavy chain portion contains an additional few amino acids, including cysteine residues that form disulfide linkages joining the two fragments.

As used herein, a “variable domain” with reference to an antibody is a specific immunoglobulin (Ig) domain of an antibody heavy or light chain that contains a sequence of amino acids that varies among different antibodies. Each light chain and each heavy chain has one variable region domain (V_{L}, and, V_{H}). The variable domains provide antigen specificity, and thus are responsible for antigen recognition. Each variable region contains CDRs that are part of the antigen binding site domain and framework regions (FRs).

Hence, an “antibody or portion thereof that is sufficient to form an antigen binding site” includes that the antibody or portion thereof contains at least 1 or 2, typically 3, 4, 5 or all 6 CDRs of the V_{H} and V_{L} sufficient to retain at least a portion of the binding specificity of the corresponding full-length antibody containing all 6 CDRs. Generally, a sufficient antigen binding site at least requires CDR3 of the heavy chain (CDRH3). It typically further requires the CDR3 of the light chain (CDRL3). As described herein, one of skill in the art knows and can identify the CDRs based on Kabat or Chothia numbering (see, e.g., Kabat, E. A. et al. (1991) Sequences of Proteins of Immunological Interest, Fifth Edition, U.S. Department of Health and Human Services, NIH Publication No. 91-3242, and Chothia et al. (1987) J. Mol. Biol. 196:901-917).

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

As used herein, the terms “antigen” and like terms are used herein to refer to a molecule, compound, or complex that is recognized by an antibody, conjugate thereof (e.g., chimeric or bispecific antibodies or scFv's), or fragment thereof (e.g., Fab, F(ab′)2, Fv, scFv, Fd, dAb and other compositions). The term can refer to any molecule that can be specifically recognized by an antibody, conjugate thereof or fragment thereof, e.g., a peptide, polynucleotide, carbohydrate, lipid, chemical moiety, or combinations thereof (e.g., phosphorylated or glycosylated peptides, chromatin moieties, etc.).

The terms “specific for,” “specifically binds,” and the like refer to the binding of a molecule (e.g., antibody or antibody fragment) to a target (antigen, epitope, antibody target, etc.) with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, a Fab fragment that specifically binds, or is specific for, is a Fab fragment that will typically bind its target antigen with at least a 2-fold greater affinity than a non-target antigen.

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., 75, 80, 85, 90, 91, 92, 93, 94, 95, 96, 97, 98, 99, or 100%). One of skill will recognize that some variability will arise depending on the method and/or threshold of determining binding.

As used herein, the term “binding affinity” refers to the strength of binding between the binding site (of a Fab fragment) and a target molecule (target antigen). The affinity of a binding site X for a target molecule Y is represented by the dissociation constant (K_{d}), which is the concentration of Y that is required to occupy half of the binding sites of X present in a solution. A lower K_{d} indicates a stronger or higher-affinity interaction between X and Y and a lower concentration of ligand is needed to occupy the sites.

The term “nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. The term can also be used interchangeably with the term “nucleic acid sequence” which includes the specific order of nucleotides in a linear sequence of nucleotides that can be transcribed and translated into an amino acid sequence of a protein of interest. Thus, the term includes nucleic acids that encode a heavy chain polypeptide and/or a light chain polypeptide.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Unless explicitly stated otherwise, the terms “yield,” and “titer” refer to the amount of antibody produced (including the total amount of HC and LC) relative to the reaction mixture volume. For example, 200 mg/L refers to 200 mg of antibody produced per liter of reaction mixture.

The term “E. coli strain,” refers to a subtype of E. coli, the cells of which have a certain biological form and share certain genetic makeup. The term “E. coli strain having oxidative cytoplasm,” refers to an E. coli strain where some or all of the cells derived from the strain each have an oxidative cytoplasm.

The term “yield per unit volume” refers to the amount of protein (for example, an antibody) expressed or produced by a cell free synthesis system by a predetermined reaction volume. The term can include the amount (concentration) of protein expressed or produced per liter of reaction volume, for example grams per liter (g/L) or milligrams per liter (mg/L) of reaction volume.

It will be understood that all ranges described herein include the endpoint values of the range and all values in between the endpoints. For example, if the range is expressed in integers, the range can include all integer values between and including the endpoint values, and values to the first significant digit. Thus, a range of 1 to 10 includes the values 1.0, 1.1, 1.2, . . . 9.8, 9.9, and 10.0.

### DETAILED DESCRIPTION OF THE INVENTION

The instant disclosure describes compositions and methods for large scale production of antibodies using a cell-free protein synthesis system. The methods comprise expressing a heavy chain (HC) polypeptide of an antibody from a nucleic acid encoding the heavy chain in a reaction mixture comprising a light chain (LC) polypeptide. The methods are performed at a large scale that is suitable for commercial production of antibodies, for example in a reaction volume equal to or greater than about 10 liters, for example about 10 to about 10,000 liters. The yield per unit volume of antibodies produce by the methods was similar across different reaction volumes, for example in small volumes compared to large volumes, showing that the yield was scalable from reaction volumes typical of laboratory experiments to reaction volumes suitable for commercial production of antibodies.

The methods described herein unexpectedly result in increased yields per unit volume of an antibody of interest. The methods may also result in increased quality of an of antibody of interest. By increased quality, it is understood that the HC and LC polypeptide of antibodies need to be properly folded and assembled to form a functional antigen-binding site. Thus, the methods described herein may increase the yield of properly folded and/or assembled antibody per unit volume compared to a reaction mixture where both the heavy and light chain polypeptides are expressed. For example, the yield per unit volume of properly folded antibody may be at least 30% higher compared to a reaction mixture where both the heavy and light chain polypeptides are expressed from nucleic acids encoding the HC and LC.

In some aspects, the LC polypeptide is added to the cell-free protein synthesis system reaction mixture prior to the step of expressing the HC polypeptide. In some embodiments, the light chain polypeptide is a pre-fabricated light chain (PFLC). For example, the LC polypeptide can be expressed from a nucleic acid encoding the LC polypeptide in a cell-free protein synthesis system, reaction mixture, or cell free extract that is separate or different from the cell-free protein synthesis system used to express to HC polypeptide. In other embodiments, the LC polypeptide can be expressed in a cell, e.g., an E. coli cell or a CHO cell, and purified from a culture of such cells. The PFLC polypeptide can then be added to the cell-free protein synthesis reaction mixture containing the nucleic acid encoding the HC polypeptide. In some embodiments, a portion of the cell free extract containing the expressed LC polypeptide is added to the reaction mixture containing the nucleic acid encoding the HC polypeptide prior to the expressing step. In some embodiments, the expressed LC polypeptide is purified or partially purified before being added to the cell-free protein synthesis reaction mixture containing the nucleic acid encoding the HC polypeptide.

In some or any of the embodiments described herein, the LC polypeptide is made, produced or synthesized before being added to the reaction mixture containing the nucleic acid encoding the HC polypeptide. The LC polypeptide can be made, produced or synthesized using any method known in the art. For example, the light chain polypeptide can be produced in a separate reaction before being added to the reaction mixture, or the light chain polypeptide can be synthesized or prefabricated before being added to the reaction mixture. The light chain polypeptide can also be expressed from a nucleic acid encoding the light chain polypeptide in a cell-free protein synthesis system, reaction mixture, or cell free extract. The light chain polypeptide can also expressed from a nucleic acid encoding the light chain polypeptide in an intact living cell, such as a bacterial cell or mammalian cell. The light chain polypeptide can also be purified or partially purified from cell-free protein synthesis systems, reaction mixtures, cell free extracts, and/or from cultures of cells before being added to the reaction mixture.

### General Methods

Unless defined otherwise, all technical and scientific terms used herein have the meaning commonly understood by one of ordinary skill in the art to which this invention belongs. Practitioners are particularly directed to Green, M. R. and Sambrook, J., eds., Molecular Cloning: A Laboratory Manual, 4th ed., Cold Spring Harbor Laboratory Press, Cold Spring Harbor, N.Y. (2012), and Ausubel, F. M., et al. Current Protocols in Molecular Biology (Supplement 99), John Wiley & Sons, New York (2012), which are incorporated herein by reference, for definitions and terms of the art. Standard methods also appear in Bindereif, Schón, & Westhof (2005) Handbook of RNA Biochemistry, Wiley-VCH, Weinheim, Germany which describes detailed methods for RNA manipulation and analysis, and is incorporated herein by reference. Examples of appropriate molecular techniques for generating recombinant nucleic acids, and instructions sufficient to direct persons of skill through many cloning exercises are found in Green, M. R., and Sambrook, J (Id.); Ausubel, F. M., et al. (Id.); Berger and Kimmel, Guide to Molecular Cloning Techniques, Methods in Enzymology (Volume 152 Academic Press, Inc., San Diego, Calif. 1987); and PCR Protocols: A Guide to Methods and Applications (Academic Press, San Diego, Calif. 1990), which are incorporated by reference herein.

Methods for protein purification, chromatography, electrophoresis, centrifugation, and crystallization are described in Coligan et al. (2000) Current Protocols in Protein Science, Vol. 1, John Wiley and Sons, Inc., New York. Methods for cell-free synthesis are described in Spirin & Swartz (2008) Cell-free Protein Synthesis, Wiley-VCH, Weinheim, Germany. Methods for incorporation of non-native amino acids into proteins using cell-free synthesis are described in Shimizu et al. (2006) FEBS Journal, 273, 4133-4140.

PCR amplification methods are well known in the art and are described, for example, in Innis et al. PCR Protocols: A Guide to Methods and Applications, Academic Press Inc. San Diego, Calif., 1990. An amplification reaction typically includes the DNA that is to be amplified, a thermostable DNA polymerase, two oligonucleotide primers, deoxynucleotide triphosphates (dNTPs), reaction buffer and magnesium. Typically a desirable number of thermal cycles is between 1 and 25. Methods for primer design and optimization of PCR conditions are well known in the art and can be found in standard molecular biology texts such as Ausubel et al. Short Protocols in Molecular Biology, 5^{th} Edition, Wiley, 2002, and Innis et al. PCR Protocols, Academic Press, 1990. Computer programs are useful in the design of primers with the required specificity and optimal amplification properties (e.g., Oligo Version 5.0 (National Biosciences)). In some embodiments, the PCR primers may additionally contain recognition sites for restriction endonucleases, to facilitate insertion of the amplified DNA fragment into specific restriction enzyme sites in a vector. If restriction sites are to be added to the 5′ end of the PCR primers, it is preferable to include a few (e.g., two or three) extra 5′ bases to allow more efficient cleavage by the enzyme. In some embodiments, the PCR primers may also contain an RNA polymerase promoter site, such as T7 or SP6, to allow for subsequent in vitro transcription. Methods for in vitro transcription are well known to those of skill in the art (see, e.g., Van Gelder et al. Proc. Natl. Acad. Sci. U.S.A. 87:1663-1667, 1990; Eberwine et al. Proc. Natl. Acad. Sci. U.S.A. 89:3010-3014, 1992).

When the proteins described herein are referred to by name, it is understood that this includes proteins with similar functions and similar amino acid sequences. Thus, the proteins described herein include the wild-type prototype protein, as well as homologs, polymorphic variations and recombinantly created muteins. Proteins are defined as having similar amino acid sequences if they have at least 80%, 85%, 90%, 95%, 96%, 97%, 98%, or 99% sequence identity to the prototype protein. The sequence identity of a protein can be determined using the BLASTP program with the defaults wordlength of 3, an expectation (E) of 10, and the BLOSUM62 scoring matrix (see Henikoff and Henikoff, Proc. Natl. Acad. Sci. USA 89:10915-10919, 1992).

A readily conventional test to determine if a protein homolog, polymorphic variant or recombinant mutein is inclusive of a protein described herein is by specific binding to polyclonal antibodies generated against the prototype protein.

Cell Free Protein Synthesis (CFPS) Technology

In order to express the biologically active proteins of interest described herein, a cell free protein synthesis system can be used. Cell extracts have been developed that support the synthesis of proteins in vitro from purified mRNA transcripts or from mRNA transcribed from DNA during the in vitro synthesis reaction. The cell free protein synthesis systems described herein can comprise large reaction volumes, for example reaction volumes equal to or greater than about 10 liters, such as equal to or greater than about 10, about 20, about 30, about 40, about 50, about 60, about 70, about 80, about 90, about 100, about 150, about 200, about 300, about 400, about 500, about 600, about 700, about 800, about 900, about 1000, about 2000, about 3000, about 4000, about 5000, about 6000, about 7000, about 8000, about 9000, about 10,000 liters, about 15,000 liters, about 20,000 liters, or about 25,000 liters. In some embodiments, the cell free protein synthesis systems described herein can comprise reaction volumes equal to or less than about 10 liters, such as equal to or less than about 10, about 20, about 30, about 40, about 50, about 60, about 70, about 80, about 90, about 100, about 150, about 200, about 300, about 400, about 500, about 600, about 700, about 800, about 900, about 1000, about 2000, about 3000, about 4000, about 5000, about 6000, about 7000, about 8000, about 9000, about 10,000, about 15,000, about 20,000, or about 25,000 liters. In some embodiments, the cell free protein synthesis systems described herein can comprise reaction volumes of about 10,000 to 20,000 liters, or reaction volumes equal to or less than about 10,000 to 20,000 liters, or a maximum volume of about 10,000 to 20,000 liters, for example, a maximum volume of 10,000 liters, 11,000 liters, 12,000 liters, 13,000 liters, 14,000 liters, 15,000 liters, 16,000 liters, 17,000 liters, 18,000 liters, 19,000 liters, or 20,000 liters. In some embodiments, the cell free protein synthesis systems described herein can comprise reaction volumes of about 10,000 liters, reaction volumes equal to or less than about 10,000 liters, or a maximum reaction volume of about 10,000 liters.

In some embodiments, the cell-free protein synthesis system comprises a reaction mixture having a volume of about 10 to about 25,000 liters. In some embodiments, the cell-free protein synthesis system comprises a reaction mixture having a volume of about 10 to about 10,000 liters. In some embodiments, the reaction mixture comprises a volume of about 10,000 liters to about 20,000 liters. In some embodiments, the reaction mixture comprises a volume of about 15,000 to about 25,000 liters. In some embodiments, the reaction mixture comprises a volume equal to or greater than about 10 liters to about 25,000 liters. In some embodiments, the reaction mixture comprises a volume equal to or less than about 10 liters to about 25,000 liters.

In some embodiments, the protein of interest is an antibody.

CFPS of polypeptides in a reaction mix comprises bacterial extracts and/or defined reagents. The reaction mix comprises at least ATP or an energy source; a template for production of the macromolecule, e.g., DNA, mRNA, etc.; amino acids, and such co-factors, enzymes and other reagents that are necessary for polypeptide synthesis, e.g., ribosomes, tRNA, polymerases, transcriptional factors, aminoacyl synthetases, elongation factors, initiation factors, etc. In one embodiment of the invention, the energy source is a homeostatic energy source. Also included may be enzyme(s) that catalyze the regeneration of ATP from high-energy phosphate bonds, e.g., acetate kinase, creatine kinase, etc. Such enzymes may be present in the extracts used for translation, or may be added to the reaction mix. Such synthetic reaction systems are well-known in the art, and have been described in the literature.

The term “reaction mix” as used herein, refers to a reaction mixture capable of catalyzing the synthesis of polypeptides from a nucleic acid template. The reaction mixture comprises extracts from bacterial cells, e.g, E. coli S30 extracts. S30 extracts are well known in the art, and are described in, e.g., Lesley, S. A., et al. (1991), J. Biol. Chem. 266, 2632-8. The synthesis can be performed under either aerobic or anaerobic conditions.

In some embodiments, the bacterial extract is dried. The dried bacterial extract can be reconstituted in milli-Q water (e.g., reverse osmosis water) at 110% of the original solids as determined by measuring the percent solids of the starting material. In one embodiment, an accurately weighed aliquot of dried extract, representing 110% of the original solids of 10 mL of extract, is added to 10 mL of Milli-Q water in a glass beaker with a stir bar on a magnetic stirrer. The resulting mixture is stirred until the powder is dissolved. Once dissolved, the material is transferred to a 15 mL Falcon tube and stored at −80 C unless used immediately.

The volume percent of extract in the reaction mix will vary, where the extract is usually at least about 10% of the total volume; more usually at least about 20%; and in some instances may provide for additional benefit when provided at least about 50%; or at least about 60%; and usually not more than about 75% of the total volume.

The general system includes a nucleic acid template that encodes a protein of interest. The nucleic acid template is an RNA molecule (e.g., mRNA) or a nucleic acid that encodes an mRNA (e.g., RNA, DNA) and be in any form (e.g., linear, circular, supercoiled, single stranded, double stranded, etc.). Nucleic acid templates guide production of the desired protein.

To maintain the template, cells that are used to produce the extract can be selected for reduction, substantial reduction or elimination of activities of detrimental enzymes or for enzymes with modified activity. Bacterial cells with modified nuclease or phosphatase activity (e.g., with at least one mutated phosphatase or nuclease gene or combinations thereof) can be used for synthesis of cell extracts to increase synthesis efficiency. For example, an E. coli strain used to make an S30 extract for CFPS can be RNase E or RNase A deficient (for example, by mutation).

CFPS systems can also be engineered to guide the incorporation of detectably labeled amino acids, or unconventional or unnatural amino acids, into a desired protein. The amino acids can be synthetic or derived from another biological source. Various kinds of unnatural amino acids, including without limitation detectably labeled amino acids, can be added to CFPS reactions and efficiently incorporated into proteins for specific purposes. See, for example, Albayrak, C. and Swartz, J R., Biochem. Biophys Res. Commun., 431(2):291-5; Yang W C et al. Biotechnol. Prog. (2012), 28(2):413-20; Kuechenreuther et al. PLoS One, (2012), 7(9):e45850; and Swartz J R., AIChE Journal, 58(1):5-13.

In a generic CFPS reaction, a gene encoding a protein of interest is expressed in a transcription buffer, resulting in mRNA that is translated into the protein of interest in a CFPS extract and a translation buffer. The transcription buffer, cell-free extract and translation buffer can be added separately, or two or more of these solutions can be combined before their addition, or added contemporaneously.

To synthesize a protein of interest in vitro, a CFPS extract at some point comprises a mRNA molecule that encodes the protein of interest. In some CFPS systems, mRNA is added exogenously after being purified from natural sources or prepared synthetically in vitro from cloned DNA using RNA polymerases such as RNA polymerase II, SP6 RNA polymerase, T3 RNA polymerase, T7 RNA polymerase, RNA polymerase III and/or phage derived RNA polymerases. In other systems, the mRNA is produced in vitro from a template DNA; both transcription and translation occur in this type of CFPS reaction. In some embodiments, the transcription and translation systems are coupled or comprise complementary transcription and translation systems, which carry out the synthesis of both RNA and protein in the same reaction. In such in vitro transcription and translation systems, the CFPS extracts contain all the components (exogenous or endogenous) necessary both for transcription (to produce mRNA) and for translation (to synthesize protein) in a single system. The coupled transcription and translation systems described herein are sometimes referred to as Open-Cell Free Synthesis (OCFS) systems, and are capable of achieving high titers of properly folded proteins of interest, e.g., high titers of antibody expression.

A cell free protein synthesis reaction mixture comprises the following components: a template nucleic acid, such as DNA, that comprises a gene of interest operably linked to at least one promoter and, optionally, one or more other regulatory sequences (e.g., a cloning or expression vector containing the gene of interest) or a PCR fragment; an RNA polymerase that recognizes the promoter(s) to which the gene of interest is operably linked (e.g. T7 RNA polymerase) and, optionally, one or more transcription factors directed to an optional regulatory sequence to which the template nucleic acid is operably linked; ribonucleotide triphosphates (rNTPs); optionally, other transcription factors and co-factors therefor; ribosomes; transfer RNA (tRNA); other or optional translation factors (e.g., translation initiation, elongation and termination factors) and co-factors therefore; one or more energy sources, (e.g., ATP, GTP); optionally, one or more energy regenerating components (e.g., PEP/pyruvate kinase, AP/acetate kinase or creatine phosphate/creatine kinase); optionally factors that enhance yield and/or efficiency (e.g., nucleases, nuclease inhibitors, protein stabilizers, chaperones) and co-factors therefore; and; optionally, solubilizing agents. The reaction mix further comprises amino acids and other materials specifically required for protein synthesis, including salts (e.g., potassium, magnesium, ammonium, and manganese salts of acetic acid, glutamic acid, or sulfuric acids), polymeric compounds (e.g., polyethylene glycol, dextran, diethyl aminoethyl dextran, quaternary aminoethyl and aminoethyl dextran, etc.), cyclic AMP, inhibitors of protein or nucleic acid degrading enzymes, inhibitors or regulators of protein synthesis, oxidation/reduction adjuster (e.g., DTT, ascorbic acid, glutathione, and/or their oxides), non-denaturing surfactants (e.g., Triton X-100), buffer components, spermine, spermidine, putrescine, etc. Components of CFPS reactions are discussed in more detail in U.S. Pat. Nos. 7,338,789 and 7,351,563, and U.S. App. Pub. Nos. 2010/0184135 and US 2010/0093024, the disclosures of each of which is incorporated by reference in its entirety for all purposes.

Depending on the specific enzymes present in the extract, for example, one or more of the many known nuclease, polymerase or phosphatase inhibitors can be selected and advantageously used to improve synthesis efficiency.

Protein and nucleic acid synthesis typically requires an energy source. Energy is required for initiation of transcription to produce mRNA (e.g., when a DNA template is used and for initiation of translation high energy phosphate for example in the form of GTP is used). Each subsequent step of one codon by the ribosome (three nucleotides; one amino acid) requires hydrolysis of an additional GTP to GDP. ATP is also typically required. For an amino acid to be polymerized during protein synthesis, it must first be activated. Significant quantities of energy from high energy phosphate bonds are thus required for protein and/or nucleic acid synthesis to proceed.

An energy source is a chemical substrate that can be enzymatically processed to provide energy to achieve desired chemical reactions. Energy sources that allow release of energy for synthesis by cleavage of high-energy phosphate bonds such as those found in nucleoside triphosphates, e.g., ATP, are commonly used. Any source convertible to high energy phosphate bonds is especially suitable. ATP, GTP, and other triphosphates can normally be considered as equivalent energy sources for supporting protein synthesis.

To provide energy for the synthesis reaction, the system can include added energy sources, such as glucose, pyruvate, phosphoenolpyruvate (PEP), carbamoyl phosphate, acetyl phosphate, creatine phosphate, phosphopyruvate, glyceraldehyde-3-phosphate, 3-phosphoglycerate and glucose-6-phosphate, that can generate or regenerate high-energy triphosphate compounds such as ATP, GTP, other NTPs, etc.

When sufficient energy is not initially present in the synthesis system, an additional source of energy is preferably supplemented. Energy sources can also be added or supplemented during the in vitro synthesis reaction.

In some embodiments, the cell-free protein synthesis reaction is performed using the PANOx-SP system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, phosphoenol pyruvate (PEP), NAD, CoA, Na⁺ oxalate, putrescine, spermidine, and S30 extract.

In some embodiments, proteins containing a non-natural amino acid (nnAA) may be synthesized. In such embodiments, the reaction mix may comprise the non-natural amino acid, a tRNA orthogonal to the 20 naturally occurring amino acids, and a tRNA synthetase that can link the nnAA with the orthogonal tRNA. See, e.g., US Pat. App. Pub. No. US 2010/0093024. Alternately, the reaction mix may comprise a nnAA conjugated to a tRNA for which the naturally occurring tRNA synthetase has been depleted. See, e.g., PCT Pub. No. WO2010/081111. The nnAA can be selected from any nnAA known in the art. For example, the nnAA can be selected from those described in U.S. Pat. No. 9,738,724 and U.S. Pat. No. 10,610,571. In some embodiments, the nnAA is p-acetyl-phenylalanine. In some embodiments, the nnAA is p-amino-methyl-phenylalanine. In some embodiments, the nnAA is p-azidomethyl-L-phenylalanine.

In some instances, the cell-free synthesis reaction does not require the addition of commonly secondary energy sources, yet uses co-activation of oxidative phosphorylation and protein synthesis. In some instances, CFPS is performed in a reaction such as the Cytomim (cytoplasm mimic) system. The Cytomim system is defined as a reaction condition performed in the absence of polyethylene glycol with optimized magnesium concentration. This system does not accumulate phosphate, which is known to inhibit protein synthesis.

The presence of an active oxidative phosphorylation pathway can be tested using inhibitors that specifically inhibit the steps in the pathway, such as electron transport chain inhibitors. Examples of inhibitors of the oxidative phosphorylation pathway include toxins such as cyanide, carbon monoxide, azide, carbonyl cyanide m-chlorophenyl hydrazone (CCCP), and 2,4-dinitrophenol, antibiotics such as oligomycin, pesticides such as rotenone, and competitive inhibitors of succinate dehydrogenase such as malonate and oxaloacetate.

In some embodiments, the cell-free protein synthesis reaction is performed using the Cytomim system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, sodium pyruvate, NAD, CoA, Na⁺ oxalate, putrescine, spermidine, and S30 extract. In some embodiments, the energy substrate for the Cytomim system is pyruvate, glutamic acid, and/or glucose. In some embodiments of the system, the nucleoside triphosphates (NTPs) are replaced with nucleoside monophosphates (NMPs).

The cell extract can be treated with iodoacetamide in order to inactivate enzymes that can reduce disulfide bonds and impair proper protein folding. As further described herein, the cell extract can also be supplemented with a chaperone that promotes proper protein folding. In some embodiments, the chaperone is a protein disulfide isomerase (PDI) or a peptidyl prolyl isomerase (PPI). Thus, the cell extract can be supplemented with a PDI such as but not limited to E. coli DsbC, or a PPI such as but not limited to FkpA, or a combination of a PDI and PPI. Examples of suitable chaperones are further described herein. Glutathione disulfide (GSSG) and glutathione (GSH) can also be added to the extract at a ratio that promotes proper protein folding and prevents the formation of aberrant protein disulfides.

In some embodiments, the CFPS reaction includes inverted membrane vesicles to perform oxidative phosphorylation. These vesicles can be formed during the high pressure homogenization step of the preparation of cell extract process, as described herein, and remain in the extract used in the reaction mix.

The cell-free extract can be thawed to room temperature before use in the CFPS reaction. The extract can be incubated with 50 μM iodoacetamide for 30 minutes when synthesizing protein with disulfide bonds. In some embodiments, the CFPS reaction includes about 30% (v/v) iodoacetamide-treated extract with about 8 mM magnesium glutamate, about 10 mM ammonium glutamate, about 130 mM potassium glutamate, about 35 mM sodium pyruvate, about 1.2 mM AMP, about 0.86 mM each of GMP, UMP, and CMP, about 2 mM amino acids (about 1 mM for tyrosine), about 4 mM sodium oxalate, about 0.5 mM putrescine, about 1.5 mM spermidine, about 16.7 mM potassium phosphate, about 100 mM T7 RNA polymerase, about 2-10 μg/mL plasmid DNA template, about 1-10 μM E. coli DsbC, and a total concentration of about 2 mM oxidized (GSSG) glutathione. Optionally, the cell free extract can include 1 mM of reduced (GSH).

The cell free synthesis reaction conditions may be performed as batch, continuous flow, or semi-continuous flow, as known in the art. The reaction conditions are linearly scalable, for example, from about the 0.3 L scale in about a 0.5 L stirred tank reactor, to about the 4 L scale in about a 10 L reactor, to about the 100 L scale in about a 200 L reactor, to about the 200 L scale in about a 500 L reactor, to about the 500 L scale in about a 1000 L reactor, to about the 1000 L scale in about a 2000 L reactor, to about the 2000 L scale in about a 4000 L reactor, to about the 3000 L scale in about a 6000 L reactor, to about the 4000 L scale in about a 8000 L reactor, to about the 5000 L scale in about a 10,000 L reactor, to about the 6000 L scale in about a 12,000 L reactor, to about the 7000 L scale in about a 14,000 L reactor, to about the 8000 L scale in about a 16,000 L reactor, to about the 9000 L scale in about a 18,000 L reactor, to about the 10,000 L scale in about a 20,000 L reactor, to about the 20,000 L scale in about a 40,000 L reactor, and to about the 25,000 L scale in about a 50,000 L reactor.

The cell free synthesis reaction conditions can comprise reaction volumes equal to or greater than about 0.3 liters, about 1 liter, about 5 liters, about 10 liters, such as equal to or greater than about 10, about 20, about 30, about 40, about 50, about 60, about 70, about 80, about 90, about 100, about 150, about 200, about 300, about 400, about 500, about 600, about 700, about 800, about 900, about 1000, about 2000, about 3000, about 4000, about 5000, about 6000, about 7000, about 8000, about 9000, about 10,000 liters, about 15,000 liters, about 20,000 liters, or about 25,000 liters. In some embodiments, the cell free synthesis reaction conditions comprise reaction volumes equal to or less than about 0.3, about 1.0, about 5, about 10, about 20, about 30, about 40, about 50, about 60, about 70, about 80, about 90, about 100, about 150, about 200, about 300, about 400, about 500, about 600, about 700, about 800, about 900, about 1000, about 2000, about 3000, about 4000, about 5000, about 6000, about 7000, about 8000, about 9000, about 10,000, about 15,000, about 20,000, or equal to or less than about 25,000 liters. In some embodiments, the cell free synthesis reaction conditions can comprise reaction volumes of about 10,000 to 25,000 liters, or equal to or less than about 10,000 to 25,000 liters, or a maximum volume of about 10,000 to 25,000 liters, for example, a maximum volume of 10,000 liters, 11,000 liters, 12,000 liters, 13,000 liters, 14,000 liters, 15,000 liters, 16,000 liters, 17,000 liters, 18,000 liters, 19,000 liters, 20,000 liters, 21,000 liters, 22,000 liters, 23,000 liters 24,000 liters, or 25,000 liters. In some embodiments, the cell free synthesis reaction conditions can comprise reaction volumes of about 10 to about 25,000 liters, about 10 to about 10,000 liters, about 10,000 liters to about 20,000 liters, or about 15,000 liters to about 25,000 liters.

The development of a continuous flow in vitro protein synthesis system by Spirin et al. (1988) Science 242:1162-1164 proved that the reaction could be extended up to several hours. Since then, numerous groups have reproduced and improved this system (see, e.g., Kigawa et al. (1991) J. Biochem. 110:166-168; Endo et al. (1992) J. Biotechnol. 25:221-230). Kim and Choi (Biotechnol. Prog. 12: 645-649, 1996) have reported that the merits of batch and continuous flow systems can be combined by adopting a “semicontinuous operation” using a simple dialysis membrane reactor. They were able to reproduce the extended reaction period of the continuous flow system while maintaining the initial rate of a conventional batch system. However, both the continuous and semi-continuous approaches require quantities of expensive reagents, which must be increased by a significantly greater factor than the increase in product yield.

Several improvements have been made in the conventional batch system (Kim et al. (1996) Eur. J. Biochem. 239: 881-886; Kuldlicki et al. (1992) Anal. Biochem. 206:389-393; Kawarasaki et al. (1995) Anal. Biochem. 226: 320-324). Although the semicontinuous system maintains the initial rate of protein synthesis over extended periods, the conventional batch system still offers several advantages, e.g. convenience of operation, easy scale-up, lower reagent costs and excellent reproducibility. Also, the batch system can be readily conducted in multiplexed formats to express various genetic materials simultaneously.

Patnaik and Swartz (Biotechniques 24:862-868, 1998) have reported that the initial specific rate of protein synthesis could be enhanced to a level similar to that of in vivo expression through extensive optimization of reaction conditions. It is notable that they achieved such a high rate of protein synthesis using the conventional cell extract prepared without any condensation steps (Nakano et al. (1996) J. Biotechnol. 46:275-282; Kim et al. (1996) Eur. J. Biochem. 239:881-886). Kigawa et al. (1999) FEBS Lett 442:15-19 report high levels of protein synthesis using condensed extracts and creatine phosphate as an energy source. These results imply that further improvement of the batch system, especially in terms of the longevity of the protein synthesis reaction, would substantially increase the productivity for batch in vitro protein synthesis. However, the reason for the early halt of protein synthesis in the conventional batch system has remained unclear.

The protein synthesis reactions described herein can utilize a large scale reactor, small scale, or may be multiplexed to perform a plurality of simultaneous syntheses. In some embodiments, the protein synthesis reactions described are continuous reactions that use a feed mechanism to introduce a flow of reagents, and may isolate the end-product as part of the process. In some embodiments, the protein synthesis reactions described herein are batch reactions, where additional reagents may be introduced to prolong the period of time for active synthesis. A reactor can be run in any mode such as batch, extended batch, semi-batch, semi-continuous, fed-batch and continuous, and which will be selected in accordance with the application purpose.

Generating a Lysate

The methods and systems described herein use a cell lysate for in vitro translation of a target protein of interest. For convenience, the organism used as a source for the lysate may be referred to as the source organism or host cell. Host cells may be bacteria, yeast, mammalian or plant cells, or any other type of cell capable of protein synthesis. A lysate comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-1, IF-2, and IF-3, elongation factors such as EF-Tu, EF-Ts, and EF-G, release factors such as RF-1, RF-2, and RF-3, and the like.

An embodiment uses a bacterial cell from which a lysate is derived. A bacterial lysate derived from any strain of bacteria can be used in the methods of the invention. The bacterial lysate can be obtained as follows. The bacteria of choice are grown to log phase in any of a number of growth media and under growth conditions that are well known in the art and easily optimized by a practitioner for growth of the particular bacteria. For example, a natural environment for synthesis utilizes cell lysates derived from bacterial cells grown in medium containing glucose and phosphate, where the glucose is present at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and undefined sources of nutrients. Cells that have been harvested overnight can be lysed by suspending the cell pellet in a suitable cell suspension buffer, and disrupting the suspended cells by sonication, breaking the suspended cells in a French press, continuous flow high pressure homogenization, or any other method known in the art useful for efficient cell lysis The cell lysate is then centrifuged or filtered to remove large DNA fragments and cell debris.

The bacterial strain used to make the cell lysate generally has reduced nuclease and/or phosphatase activity to increase cell free synthesis efficiency. For example, the bacterial strain used to make the cell free extract can have mutations in the genes encoding the nucleases RNase E and RNase A. The strain may also have mutations to stabilize components of the cell synthesis reaction such as deletions in genes such as tnaA, speA, sdaA or gshA, which prevent degradation of the amino acids tryptophan, arginine, serine and cysteine, respectively, in a cell-free synthesis reaction. Additionally, the strain may have mutations to stabilize the protein products of cell-free synthesis such as knockouts in the proteases ompT or lonP.

In some embodiments, the bacteria extract can be thawed to room temperature before use in the CFPS reaction. The extract can be incubated with 50 μM iodoacetamide for 30 minutes when synthesizing protein with disulfide bonds. In some embodiments, the CFPS reaction includes about 30% (v/v) iodoacetamide-treated extract with about 8 mM magnesium glutamate, about 10 mM ammonium glutamate, about 130 mM potassium glutamate, about 35 mM sodium pyruvate, about 1.2 mM AMP, about 0.86 mM each of GMP, UMP, and CMP, about 2 mM amino acids (about 1 mM for tyrosine), about 4 mM sodium oxalate, about 0.5 mM putrescine, about 1.5 mM spermidine, about 16.7 mM potassium phosphate, about 100 mM T7 RNA polymerase, about 2-10 μg/mL plasmid DNA template, about 1-10 μM E. coli DsbC, and a total concentration of about 2 mM oxidized (GSSG) glutathione. Optionally, the cell free extract can include 1 mM of reduced (GSH) glutathione.

Proteins of Interest

The methods and systems described herein are useful for increasing the expression of properly folded, biologically active proteins of interest. The protein of interest can be any protein that is capable of being expressed in a bacterial cell free synthesis system. Non-limiting examples include proteins with disulfide bonds and proteins with at least two proline residues. The protein of interest can be, for example, an antibody or fragment thereof, therapeutic proteins, growth factors, receptors, cytokines, enzymes, ligands, etc. Additional examples of proteins of interest are described below.

Antibodies

The methods provided herein can be used for recombinant production of any antibodies in a cell-free synthesis system. Polynucleotides encoding the HC of the antibody can be introduced into the cell-free synthesis system to express the HC, which can then pair (dimerize) with the LC to produce the properly assembled antibody. Disulfide bonds are present in antibodies and thus the present systems are beneficial to prevent degradation and increase yield of the antibody. Any antibodies can be produced using the method described herein in a yield, referring to the amount of protein per liter of culture medium, that is at least about 200 mg/L, or at least about 250 mg/L, at least about 500 mg/L, at least about 750 mg/L, or at least about 1000 mg/L. In some embodiments, the methods described herein yield about 200 mg/L to about 1000 mg/L of an antibody, e.g., about 200, 300, 400, 500, 600, 700, 800, 900 or 1000 mg/L of an antibody.

In some embodiments, the antibody is a monoclonal antibody, for example a monoclonal IgG antibody. In some embodiments, the antibody is a bispecific antibody comprising two Fab arms and one Fc domain, where each Fab binds a different antigen. In some embodiments, the antibody comprises one or more functional antigen binding sites, where each antigen binding site comprises a pair of heavy and light chain variable domains. The pair of heavy and light chain variable domains can be derived from a native or cognate pair of heavy and light chain variable domains expressed by a single B cell or B cell clone. Alternatively, the pair of heavy and light chain variable domains can include non-cognate or randomly paired heavy and light chain variable domains.

The antibody can also be conjugated to a therapeutic molecule, agent or drug. Thus, in some embodiments, the antibody can be used to make an antibody-drug conjugate (ADC). In some embodiments, the drug is a cytotoxic or anti-cancer drug. ADCs typically comprise a linker between the antibody and the agent or drug. Examples of suitable linkers include cleavable and non-cleavable linkers. Cleavable linkers can be designed to release the agent or drug from the ADC complex by enzymes within the target cell. If the drug is a cytotoxic or anti-cancer drug, this allows the released drug to target near-by cancer cells (so called “bystander killing”). Non-cleavable linkers are typically designed not to be released from the ADC complex after entry into a target cell. Suitable cleavable linkers comprise disulfides, hydrazones or peptides, and suitable non-cleavable linkers can comprise thioethers.

In some embodiments, the antibody is a bispecific antibody comprising a heterodimeric Fc region. For example, the antibody can be an antibody where the heterodimeric Fc region comprises two asymmetric CH3 domains that include sequences from IgA and IgG CH3 domains (see U.S. Pat. No. 9,505,848 B2 to Davis J., et al./Merck Patent GmbH; Davis, J. H et al., SEEDbodies: fusion proteins based on strand-exchange engineered domain (SEED) C_{H}3 heterodimers in an Fc analogue platform for asymmetric binders or immunofusions and bispecific antibodies, Protein Engineering, Design and Selection, Volume 23, Issue 4, April 2010, Pages 195-202); The antibody comprising a heterodimeric Fc region can be monovalent or bivalent, and can be expressed as a fusion molecule with a Fab, scFv, a Fab/scFv combination, or with a camelid single-domain antibody fragments (VHH). See M. Muda, et al., Therapeutic assessment of SEED: a new engineered antibody platform designed to generate mono- and bispecific antibodies, Protein Engineering, Design and Selection, Volume 24, Issue 5, May 2011, Pages 447-454.

In some embodiments, the antibody is a domain-exchanged antibody comprising a light chain (LC), and a heavy chain (HC), wherein the LC dimerizes with the HC. In some embodiments, the antibody is a domain-exchanged antibody comprising a light chain (LC) comprising a VL-CH3, and a heavy chain (HC) comprising VH-CH3-CH2-CH3, wherein the VL-CH3 of the LC dimerizes with the VH-CH3 of the HC, thereby forming a domain-exchanged LC/HC dimer comprising a CH3_{LC}/CH3_{HC} domain pair. Examples of such domain-exchanged antibodies are described in U.S. Patent Publication 2018/0016354 to WOZNIAK-KNOPP, G. et al./Merck Patent GmbH.

In some embodiments, the antibody is a bispecific antibody comprising an engineered CH3 domain with significantly enhanced HC heterodimerization based on steric or electrostatic complementarity. In some embodiments, the antibody is a bispecific antibody comprising engineered CH3 domains to create either a “knob” or a “hole” in each heavy chain to promote heterodimerization. Examples of knobs-into-holes technology are described in Ridgway, J. B., et al., Protein Eng. 9 (1996) 617-621; Atwell et al., Journal of Molecular Biology, vol. 270 (1997), 26-35; WO 1996/027011 A1, corresponding to U.S. Pat. No. 7,642,228 B2 to Carter, P. et al./Genentech Inc.; Merchant, A. M., et al., Nature Biotech. 16 (1998) 677-681; EP1870459 A1, and Xu Y, et al., Production of bispecific antibodies in “knobs-into-holes” using a cell-free expression system. MAbs. 2015; 7(1):231-242.

In some embodiments, the antibody is a bispecific antibody comprising a FAB that binds a first antigen and an scFv that binds a second antigen. In some embodiments, the antibody is a bispecific antibody comprising a FAB that binds a first antigen on one arm and an scFv that binds a second antigen on the second arm. In some embodiments, the bispecific antibody comprises complementary mutations in the Fc and/or heavy chain that enhance heterodimerization. Examples of such complementary mutations include T350V, L351Y, F405A, Y407V and T350V, T366L, K392 L, T394W. Representative examples of such bispecific antibodies (referred to as “bipods”) are described in Nesspor, T. C., et al., High-Throughput Generation of Bipod (Fab×scFv) Bispecific Antibodies Exploits Differential Chain Expression and Affinity Capture, Sci Rep 10, 7557 (2020), and references cited therein.

In some embodiments, the bispecific antibody comprises a scFv fused to the light or heavy chain of a Fab fragment. In some embodiments, the bispecific antibody comprises a scFv fused to the C-terminus of the light or heavy chain of a Fab fragment. For a general review of bispecific antibody formats, see Brinkmann, U. and Kontermann, R. E., The making of bispecific antibodies, mAbs, Vol. 9, 2017, 182-212.

Exemplary antibodies produced by the methods described herein are described in US 2017/0253656 A1 (anti-cluster of differentiation 74 (CD74) antibodies); US 2019/0233512 A1 (anti-folate receptor alpha (FOLR1) antibodies) and WO 2019/190969 (corresponding to U.S. Provisional Patent App. No. 62/648,266) (anti-B-cell maturation antigen (BCMA) antibodies).

In some aspects, the antibody is a SEEDbody. In some embodiments, the SEEDbody comprises an anti-EGFR-Fab-GA arm comprising a HC and LC paired with an anti-Muc1-scFv-AG arm.

In some embodiments, the antibody is selected from the group consisting of a B10 antibody (an anti folate receptor alpha antibody), an H01 antibody (an anti folate receptor alpha antibody), a 7209 antibody (anti-CD74 antibody), an anti-PD1 antibody, an anti-Tim3 antibody, an anti-HER2 antibody (e.g., trastuzumab), an anti LAG3 antibody, an anti-B cell maturation antigen (anti-BCMA) antibody, an anti-Cluster of Differentiation 74 (anti-CD74) antibody, an anti-folate receptor alpha (FOLR1) antibody, or a Seedbody. Non-limiting examples of the antibodies are listed in Table 2.

TABLE 2
Exemplary antibodies

| Antibody name | HC (SEQ ID NO) | LC (SEQ ID NO) |
|---|---|---|
| B10 | 1 | 2 |
| H01 | 17 | 2 |
| 7209 | 18 | 9 |
| anti-PD1 | 11 | 12 |
| anti-Tim3 | 13 | 14 |
| anti LAG3 | 15 | 16 |
| Trastuzumab | 20 | 2 |
| anti-CD74 | | |
| anti- FOLR1 | | |
| anti- BCMA | | |

I. Promoters

Promoters that can be used in the methods of the invention to drive the transcription of the HC may be any appropriate promoter sequence suitable for E. coli. Such promoters may include mutant, truncated, and hybrid promoters, and may be obtained from polynucleotides encoding extracellular or intracellular polypeptides either endogenous (native) or heterologous (foreign) to the cell. A promoter used herein may be a constitutive or an inducible promoter.

In some embodiments, the promoter is a constitutive promoter. The promoter may be one that has substantially the same promoter strength as T7, i.e., the strength of the promoter is at least 60%, at least 70%, at least 80% of the strength of T7 promoter. In some embodiments, the T7 promoter comprises or consists of a sequence of SEQ ID NO: 19. In some embodiments, the promoter may exhibit a strength that is within the range of 50-200%, e.g., 80%-150%, or 90%-140%, of the strength of T7 promoter. Non-limiting examples of promoters that are suitable for driving the transcription of the HC in E. coli cells include a T3 promoter, an SP6 promoter, a pBad, an XylA, or a PhoA promoter.

II. Vectors

Polynucleotides encoding an LC and an HC of the antibody can be inserted into one or two replicable vectors for expression in the E. coli. Many vectors are available for this purpose, and one of skilled in the art can readily select suitable vectors for use in the methods disclosed herein. Besides the gene of interest and promoter that drives the expression of the gene, the vector typically comprises one or more of the following: a signal sequence, an origin of replication, one or more marker genes.

Chaperones

To improve the expression of a biologically active protein of interest, the present methods and systems can use a bacterial extract comprising an exogenous protein chaperone. Molecular chaperones are proteins that assist the non-covalent folding or unfolding and the assembly or disassembly of other macromolecular structures. One major function of chaperones is to prevent both newly synthesized polypeptide chains and assembled subunits from aggregating into nonfunctional structures. The first protein chaperone identified, nucleoplasmin, assists in nucleosome assembly from DNA and properly folded histones. Such assembly chaperones aid in the assembly of folded subunits into oligomeric structures. Chaperones are concerned with initial protein folding as they are extruded from ribosomes, intracellular trafficking of proteins, as well as protein degradation of misfolded or denatured proteins. Although most newly synthesized proteins can fold in absence of chaperones, a minority strictly requires them. Typically, inner portions of the chaperone are hydrophobic whereas surface structures are hydrophilic. The exact mechanism by which chaperones facilitate folding of substrate proteins is unknown, but it is thought that by lowering the activation barrier between the partially folded structure and the native form, chaperones accelerate the desired folding steps to ensure proper folding. Further, specific chaperones unfold misfolded or aggregated proteins and rescue the proteins by sequential unfolding and refolding back to native and biologically active forms.

A subset of chaperones that encapsulate their folding substrates are known as chaperonins (e.g., Group I chaperonin GroEL/GroES complex). Group II chaperonins, for example, the TRiC (TCP-1 Ring Complex, also called CCT for chaperonin containing TCP-1) are thought to fold cytoskeletal proteins actin and tubulin, among other substrates. Chaperonins are characterized by a stacked double-ring structure and are found in prokaryotes, in the cytosol of eukaryotes, and in mitochondria.

Other types of chaperones are involved in membrane transport in mitochondria and endoplasmic reticulum (ER) in eukaryotes. Bacterial translocation-specific chaperone maintains newly synthesized precursor polypeptide chains in a translocation-competent (generally unfolded) state and guides them to the translocon, commonly known as a translocator or translocation channel. A similar complex of proteins in prokaryotes and eukaryotes most commonly refers to the complex that transports nascent polypeptides with a targeting signal sequence into the interior (cisternal or lumenal) space of the endoplasmic reticulum (ER) from the cytosol, but is also used to integrate nascent proteins into the membrane itself (membrane proteins). In the endoplasmic reticulum (ER) there are general chaperones (BiP, GRP94, GRP170), lectin (calnexin and calreticulin) and non-classical molecular chaperones (HSP47 and ERp29) helping to fold proteins. Folding chaperone proteins include protein disulfide isomerases (PDI, DsbA, DsbC) and peptidyl prolyl cis-trans isomerases (PPI, FkpA, SlyD, TF).

Many chaperones are also classified as heat shock proteins (Hsp) because they are highly upregulated during cellular stress such as heat shock, and the tendency to aggregate increases as proteins are denatured by elevated temperatures or other cellular stresses. Ubiquitin, which marks proteins for degradation, also has features of a heat shock protein. Some highly specific ‘steric chaperones’ convey unique structural conformation (steric) information onto proteins, which cannot be folded spontaneously. Other functions for chaperones include assistance in protein degradation, bacterial adhesin activity, and response to prion diseases linked to protein aggregation.

Enzymes known as foldases catalyze covalent changes essential for the formation of the native and functional conformations of synthesized proteins. Examples of foldases include protein disulfide isomerase (PDI), which acts to catalyze the formation of native disulfide bonds, and peptidyl prolyl cis-trans isomerase (PPI), which acts to catalyze isomerization of stable trans peptidyl prolyl bonds to the cis configuration necessary for the functional fold of proteins. The formation of native disulfides and the cis-trans isomerization of prolyl imide bonds are both covalent reactions and are frequently rate-limiting steps in the protein folding process. Recently proposed to be chaperone proteins, in stoichiometric concentrations foldases increase the reactivation yield of some denatured proteins. Other examples of chaperone proteins include deaggregases such as Skp, and the redox proteins Trr1 and Glr1.

In some embodiments, the protein chaperone can be co-expressed with another protein(s) that functions to increase the activity of the desired protein chaperone. For example, the Dsb proteins DsbA and DsbC can be coexpressed with DsbB and DsbD, which oxidize and reduce DsbA and DsbC, respectively.

Transforming Bacteria with Genes Encoding the Chaperones

The bacterial extracts used in the methods and systems described herein can contain one or more exogenous protein chaperone(s). The exogenous protein chaperone can be added to the extract, or can be expressed by the bacteria used to prepare the cell free extract. In the latter embodiment, the exogenous protein chaperone can be expressed from a gene encoding the exogenous protein chaperone that is operably linked to a promoter that initiates transcription of the gene.

Promoters that may be used to express a gene encoding the exogenous protein chaperone include both constitutive promoters and regulated (inducible) promoters. The promoters may be prokaryotic or eukaryotic depending on the host. Among the prokaryotic (including bacteriophage) promoters useful for practice of this invention are lac, T3, T7, lambda Pr′P1′ and trp promoters. Among the eukaryotic (including viral) promoters useful for practice of this invention are ubiquitous promoters (e.g. HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells), promoters which respond to a stimulus (e.g. steroid hormone receptor, retinoic acid receptor), tetracycline-regulated transcriptional modulators, cytomegalovirus immediate-early, retroviral LTR, metallothionein, SV-40, Ela, and MLP promoters. Tetracycline-regulated transcriptional modulators and CMV promoters are described in WO 96/01313, U.S. Pat. Nos. 5,168,062 and 5,385,839, the entire disclosures of which are incorporated herein by reference.

In some embodiments, the promoter is a constitutive promoter. Examples of constitutive promoters in bacteria include the spc ribosomal protein operon promotor P_{spc}, the β-lactamase gene promotor P_{bla} of plasmid pBR322, the P_{L} promoter of phage λ, the replication control promoters P_{RNAI} and P_{RNAII} of plasmid pBR322, the P1 and P2 promoters of the rrnB ribosomal RNA operon, the tet promoter, and the pACYC promoter.

Examples of suitable chaperones and methods for using and producing same are described in U.S. Pat. No. 10,190,145 to Yam et al.

Modified Proteins Containing an OmpT1 Protease Cleavage Site

In some aspects, the cell-free synthesis system described herein comprises a modified protein that comprises an Outer Membrane Protein T1 (OmpT1) protease cleavage site. Including the modified protein comprising an OmpT1 protease cleavage site in the cell-free synthesis systems can increase the yield of antibodies comprising non-natural amino acids at an amber codon. For example, Release Factor 1 (RF1) is part of the termination complex, and recognizes the UAG (amber) stop codon. RF1 recognition of the amber codon can promote pre-mature chain termination at the site of nnAA incorporation, which reduces the yield of desired proteins, such as antibodies. The yield of proteins comprising nnAA introduced at amber codons can be increased by decreasing the functional activity of RF1 in bacterial cell lysates. Thus, in some embodiments, the functional activity of RF1 is decreased by introducing OmpT1 protease cleavage sites into RF1. In some embodiments, the modified protein comprising an OmpT1 protease cleavage site is a Release Factor 1 (RF1) or Release Factor 2 (RF2) protein. Examples of modified proteins comprising OmpT1 protease cleavage sites are described in U.S. Patent Publication 2015/0259664 A1 and U.S. Pat. No. 9,650,621.

The yield of antibodies comprising nnAA can also be increased by attenuating RF1 activity by: 1) neutralizing antibody inactivation of RF1, 2) genomic knockout of RF1 (in an RF2 bolstered strain), and 3) site specific removal of RF1 using a strain engineered to express RF1 containing a protein tag for removal by affinity chromatography (Chitin Binding Domain and His Tag).

Quantitatively Measuring Protein of Interest

The quantity of the protein of interest, such as an antibody, produced by the methods and systems described herein can be determined using any method known in the art. For example, the expressed protein of interest can be purified and quantified using gel electrophoresis (e.g., PAGE), Western analysis or capillary electrophoresis (e.g., Caliper LabChip). Protein synthesis in cell-free translation reactions may be monitored by the incorporation of radiolabeled amino acids, typically, ³⁵S-labeled methionine or ¹⁴C-labeled leucine. Radiolabeled proteins can be visualized for molecular size and quantitated by autoradiography after electrophoresis or isolated by immunoprecipitation. The incorporation of recombinant His tags affords another means of purification by Ni²⁺ affinity column chromatography. Protein production from expression systems can be measured as soluble protein yield or by using an assay of enzymatic or binding activity.

Quantitatively Measuring Biological Activity and Proper Folding of Expressed Proteins

The biological activity of a protein of interest produced by the methods described herein can be quantified using an in vitro or in vivo assay specific for the protein of interest. The biological activity of the protein of interest can be expressed as the biological activity per unit volume of the cell-free protein synthesis reaction mixture. The proper folding of an expressed protein of interest can be quantified by comparing the amount of total protein produced to the amount of soluble protein. For example, the total amount of protein and the soluble fraction of that protein produced can be determined by radioactively labeling the protein of interest with a radiolabeled amino acid such as ¹⁴C-leucine, and precipitating the labeled proteins with TCA. The amount of folded and assembled protein can be determined by gel electrophoresis (PAGE) under reducing and non-reducing conditions to measure the fraction of soluble proteins that are migrating at the correct molecular weight. Under non-reducing conditions, protein aggregates can be trapped above the gel matrix or can migrate as higher molecular weight smears that are difficult to characterize as discrete entities, whereas under reducing conditions and upon heating of the sample, proteins containing disulfide bonds are denatured, aggregates are dissociated, and expressed proteins migrate as single bands. Methods for determining the amount of properly folded and assembled antibody proteins are described in the Examples. Functional activity of antibody molecules can be determined using an immunoassay, for example, an ELISA.

Bacterial Strains

The cell free protein synthesis system described herein can comprise a cell free extract prepared from a bacterium or bacterial strain. In some embodiments, the bacterial strain is an E. coli strain. The E. coli strain can be selected from any E. coli strain that is known to one of skill in the art. In some embodiments, the E. coli strain is a A (K-12), B, C or D strain.

### EXAMPLES

### Example 1

This example describes methods for producing different antibodies using a heavy chain expressed in the cell free synthesis reaction and a prefabricated light chain protein.

Materials and Methods

Pre-fabricated light chain protein (PFLC) was expressed in E. coli using standard recombinant protein expression and purification methods commonly known in the art. A commercially available affinity resin based on protein L was used in the purification process, but other purification methods can be used.

XpressCF+™ reactions were performed with the same method whether using PFLC or plasmid-directed expression of the light chain protein. For PFLC, the LC plasmid was omitted and the PFLC stock solution was added at an optimized concentration to maximize titer and product quality. Typically about 1.0 g/L PFLC was used in XCF reaction for maximum titer. PFLC concentrations from about 0.4 to 1.5 g/L also produced good results, but the titer benefit may not be maximal at the lower PFLC concentrations. Higher concentrations of PFLC did not continue to increase titer beyond a certain point.

General XpressCF+™ procedures are known in the art (see Zawada, J. F., et al, 2011) Microscale to manufacturing scale-up of cell-free cytokine production a new approach for shortening protein production development timelines. Biotechnol. Bioeng., 108: 1570-1578; and Groff, D., et al., (2014) Engineering toward a bacterial “endoplasmic reticulum” for the rapid expression of immunoglobulin proteins, mAbs, 6:3, 671-678.

Previously described XpressCF+™ procedures were improved by the addition of a nutrient feed during the expression process to supply additional energy source, amino acids, nucleotides, and XpressRNAP. The addition of the feed increased product titers.

Results

SP8893 (Anti-BCMA Antibody).

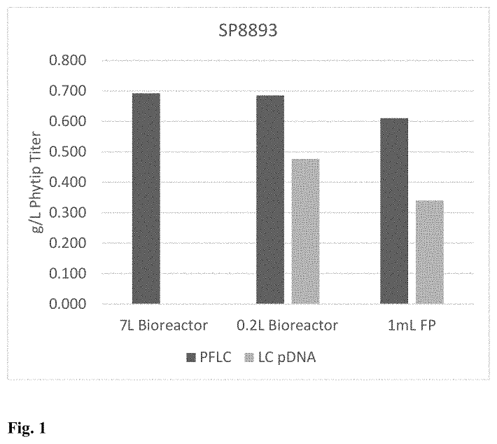

The anti-BCMA antibody was expressed in three reactor formats using PFLC: two bioreactors (7 L and 0.2 L reaction volumes) with feeding and control of pH and dissolved oxygen (DO), and a FlowerPlate® (FP) shaking plate without feeding or pH or DO control. For comparison, additional reactions were performed in the 0.2 L bioreactor and FP formats with plasmid-directed co-expression of the light chain protein (LC pDNA). The titer of anti-BCMA antibody increased about 43% in the 0.2 L bioreactor when using PFLC compared to LC pDNA. The titer in the 7 L bioreactor was essentially the same as the 0.2 L bioreactor, which demonstrates that the reaction conditions and titer benefit of PFLC were scalable. The 1 mL “FP” FlowerPlate system does not have feeding or pH control and so titers are typically lower than in bioreactors. Even so, the FP shows considerable titer improvement with the PFLC over the plasmid based system (LC pDNA). The LC pDNA system was not run in the 7 L bioreactor in this experiment. PFLC was used at 0.6 g/L in this experiment. The titer was measured using PhyTip® Columns (PhyNexus, Inc.). The results are shown in FIG. 1.

The results demonstrate that a higher titer of anti-BCMA antibody was achieved when the reactions included prefabricated light chain protein as compared to a plasmid that expressed the light chain protein.

While variability in the increase in titer using PFLC was observed under different experimental conditions, both of the experiments above demonstrate an increase in titer of anti-BCMA antibody using PFLC as compared to reactions in which the light chain protein was expressed from a plasmid.

SP8166 (Anti-Folate Receptor Alpha (FOLR1) Antibody).

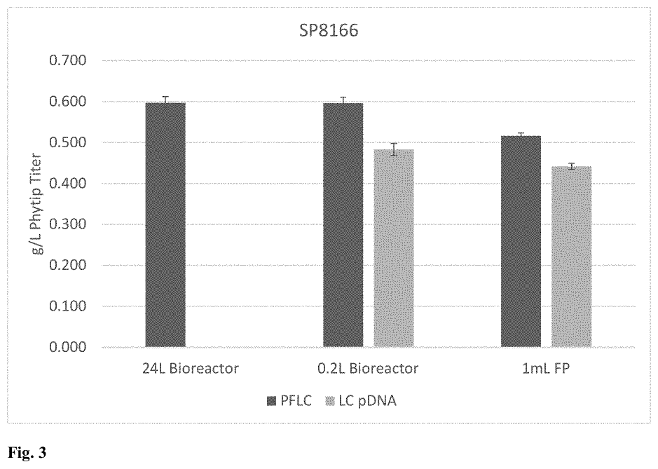

The effect of PFLC concentration was examined in 0.2 L bioreactors, also with feeding and pH and DO control, for another antibody product, SP8166 (anti-folate receptor alpha (FOLR1) antibody). The results are shown in FIG. 3.

### Results

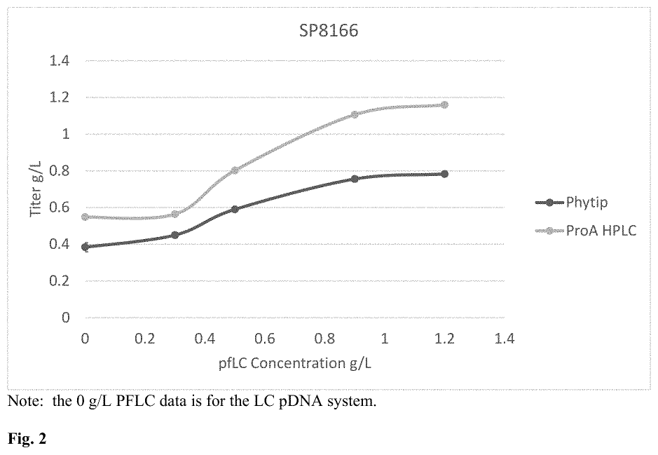

As shown in FIG. 2, the titer of anti-FOLR1 antibody increased with higher PFLC concentration up to a maximum at about 1 g/L PFLC. Titer nearly doubled when using 1.0-1.2 g/L PFLC compared to the titer with the LC pDNA system in this experiment (0 g/L PFLC in FIG. 2). The titer was measured by two methods in this experiment: PhyTip® Columns (PhyNexus, Inc.) and a protein A-based HPLC method (ProA HPLC). Both methods show similar trends in titer using a PFLC.

The titer obtained for anti-FOLR1 antibody (SP8166) using PFLC showed comparable results between 0.2 L and 24 L bioreactors (see FIG. 3). The increase in titer over the LC pDNA system was about 16% in the 0.2 L Bioreactor. The LC pDNA system was not run in the 24 L bioreactor in this experiment, and the concentration of PFLC was 0.48 g/L. An even greater increase in titer in reactions containing PFLC versus LC pDNA would be expected if higher PFLC concentrations were used in the 24 L bioreactor (see FIG. 2 for the impact of higher PFLC concentration on titer).

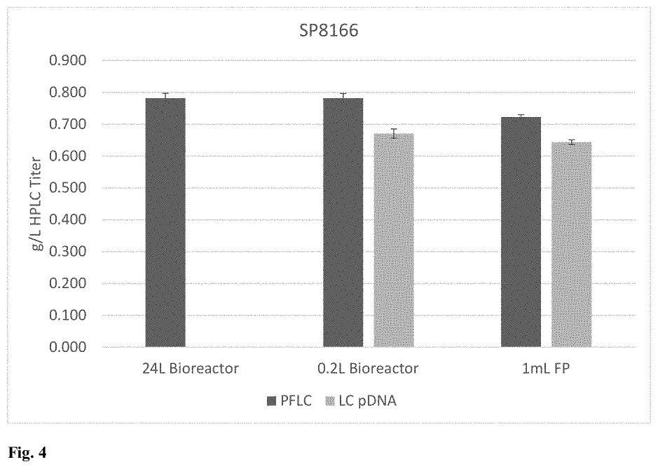

FIG. 4 shows anti-FOLR1 antibody titer data obtained by HPLC.

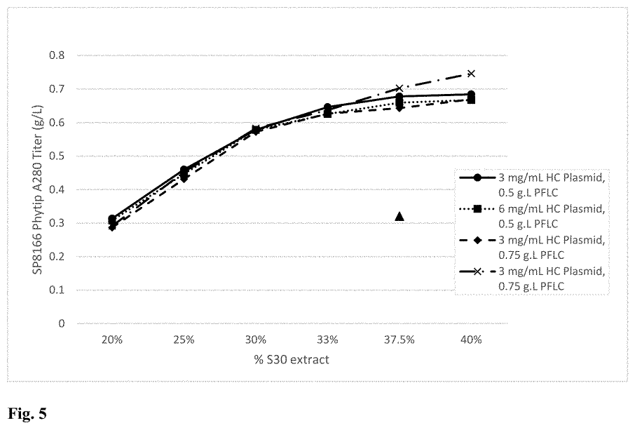

FIG. 5 shows anti-FOLR1 antibody titration data using varying amounts of XtractCF with two different concentrations of HC pDNA plasmid (3 mg/L and 6 mg/L) and PFLC (0.5 g/L and 0.75 g/L). The control was 37.5% percent of XtractCF using heavy chain and light chain expression plasmids (3 mg/L total plasmid, no PFLC).

The results in FIG. 5 demonstrate that XtractCF shows a dose-dependent increase in anti-FOLR1 antibody titer, and that the titer is substantially higher using PFLC than expressing both the heavy chain and light chains from plasmids.

SP7219 (Anti-CD74 Antibody)

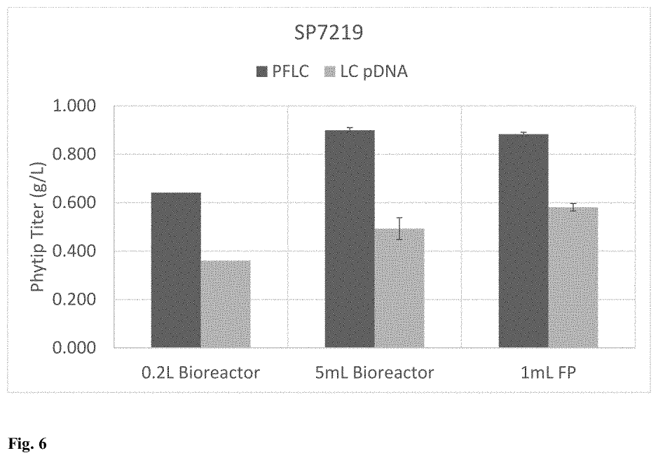

As shown in FIG. 6, PFLC increased the titer of anti-CD74 antibody about 80% in 0.2 L and 5 mL bioreactors. The PFLC reactions used 1 g/L PFLC and 6 mg/L heavy chain plasmid. The LC pDNA reactions used 3 mg/L of the plasmid encoding both the heavy and light chains. The titer increased about 50% in the 1 mL FlowerPlate format for this antibody. The data in FIG. 6 is from a batch XCF, without feeding in the reactor.

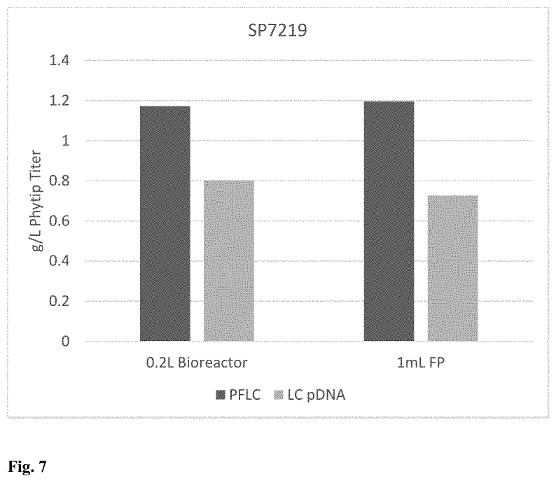

As shown in FIG. 7, XpressCF+™ using PFLC increased the titer of anti-CD74 antibody compared to XpressCF+™ extracts containing plasmid DNA encoding the light chain. The PFLC reaction used 1.25 g/L PFLC and 3.75 mg/L heavy chain plasmid. The LC pDNA reaction used a total of 5 mg/L plasmid (combined heavy chain and light chain plasmids; 0.71 mg/L LC plasmid and 4.29 mg/L HC plasmid).

In summary, the data provided in this example shows that reactions containing an expression plasmid encoding the HC and a pre-fabricated LC that was added to the reactions produced higher titers of antibodies compared to reactions containing expression plasmids encoding both the HC and LC.

### Example 2

This example demonstrates that IgG expression was increased using both purified PFLC and a cell lysate containing PFLC compared to reactions containing expression plasmids for both HC and LC.

A cell lysate of strain SBDG419 transformed with a plasmid encoding trastuzumab LC was created in buffer S30-5 (5 mM Tris-HCl pH 8.2, 1 mM Magnesium Acetate and 250 mM Potassium Acetate) at a concentration of 16.67% (w/w). Expression of Trastuzumab IgG using either the PFLC lysate reagent (4% v/v), the purified PFLC reagent (0.5 g/L) or co-expression of HC and LC was compared in a cell-free reaction with detection by C14-Leucine incorporation. Standard CF reactions are supplemented with 2% v/v L-[14C(U)]-Leucine (Perkin Elmer) and the titer was calculated by scintillation counting comparing the total counts in the reaction to counts in the acid precipitable fraction corresponding to protein synthesized (see Zawada et al., 2011). Reactions were carried out at a 100 uL scale in a 96-well plate.

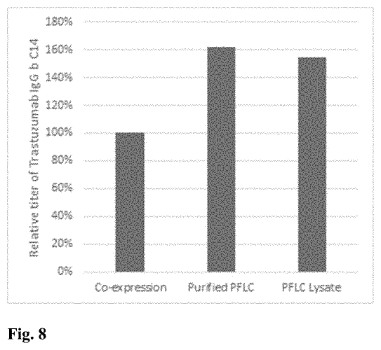

FIG. 8 shows relative expression of Trastuzuamb IgG measured by C14 Leucine incorporation comparing HC/LC co-expression to expression with purified PFLC reagent and crude PFLC lysate reagent. A similar increase in titer is observed with both the purified and crude PFLC reagents.

The data provided in this example demonstrates that IgG expression was increased using either purified PFLC or a cell lysate containing PFLC compared to reactions containing expression plasmids for both HC and LC.

### Example 3

This example demonstrates that yield of a bispecific SEEDbody was increased in reactions containing purified PFLC compared to reactions containing an expression plasmid encoding the LC.

A bispecific antibody based on the SEEDbody framework consisting of an anti-EGFR-Fab-GA arm comprised of a HC and LC paired with an anti-Muc1-scFv-AG arm (SP9203) was expressed in a small scale C14 cell free reaction to demonstrate the applicability of the PFLC strategy to a three chain bispecific antibody format (see Davis, J. H., Aperlo, C., Li, Y., Kurosawa, E., Lan, Y., Lo, K.-M., and Huston, J. S. (2010). SEEDbodies: fusion proteins based on strand-exchange engineered domain (SEED) CH3 heterodimers in an Fc analogue platform for asymmetric binders or immunofusions and bispecific antibodies. Protein Eng. Des. Sel. 23, 195-202). The three chain co-expression reaction contained plasmids encoding the HC-GA, LC and scFv-AG at total plasmid concentration of 3 ug/mL. The PFLC reagent reactions contained purified PFLC reagent at 0.5 mg/mL along with the HC-GA and scFv-AG plasmids at a total concentration of 3 ug/mL. Titer was measured by protein small scale C14 protein expression at a 100 uL scale in a 96-well plate. Standard CF reactions are supplemented with 2% v/v L-[14C(U)]-Leucine (Perkin Elmer) and the titer is calculated by scintillation counting comparing the total counts in the reaction to counts in the acid precipitable fraction corresponding to protein synthesized (see Zawada, J. F., Yin, G., Steiner, A. R., Yang, J., Naresh, A., Roy, S. M., Gold, D. S., Heinsohn, H. G., and Murray, C. J. (2011). Microscale to manufacturing scale-up of cell-free cytokine production—a new approach for shortening protein production development timelines. Biotechnol. Bioeng. 108, 1570-1578).

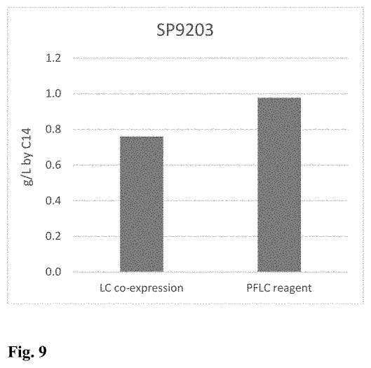

FIG. 9 shows that the yield of SEEDbody Fab/scFv bispecific antibody was increased in reactions containing the purified PFLC reagent as compared to reaction containing LC expressed from a plasmid.

The data provided in this example demonstrates that the yield of a bispecific SEEDbody was increased in reactions containing purified PFLC compared to reactions containing an expression plasmid encoding the LC.

### Example 4

This example illustrates a method for producing an anti-BCMA antibody using a heavy chain expressed in the cell free synthesis reaction and a prefabricated light chain (PFLC).

Methods:

The reaction was performed in a Micro 24 bioreactor (Micro-24 MicroReactor System from PALL® Corp.) comprising a 4 ml batch reaction and standard cell free feed (25% of the initial batch volume added). Dissolved oxygen (DO) and pH were controlled at 20% and 6.95 respectively. At 14 hrs, the DO and pH were adjusted to 80% and 8.0 respectively. The bioreactor temperature was maintained at 25° C.

The extract comprised SBDG381 extract made from a DASbox fermentation run. The heavy chain plasmid reaction concentration was 1.5, 2.15 and 2.8 mg/L. (The two plasmid system HC running concentration was 2.25 mg/L). The PreFab light chain (PFLC) concentration was 0.5, 0.875, and 1.25 g/L. The PhyTip load was 150 microL.

Results

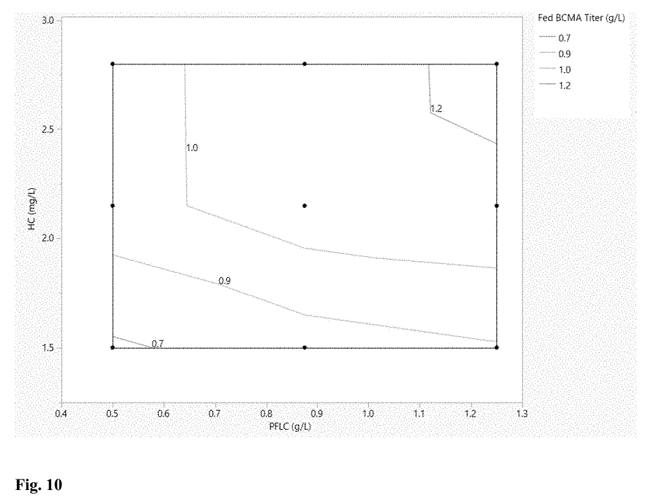

The titer of anti-BCMA antibody increased in a dose-response relationship based on the concentration of HC expression plasmid and PFLC added to the reaction. FIG. 10 shows the highest anti-BCMA antibody titer (greater than or equal to 1.2 g/L) was obtained when the concentration of both PFLC and HC were increased, i.e., to 1.25 g/L and 2.8 mg/L, respectively. A significant titer loss to below 0.7 g/L was observed by reducing PFLC and HC at the same time, i.e. to 0.5 g/L and 1.5 mg/L, respectively. For this experiment the control run (two plasmid system of heavy and light chain) expression titer was 0.615±0.011 g/L (HC concentration 2.25 mg/L; LC concentration 2.75 mg/L). Acceptable yields of anti-BCMA antibody were obtained with a minimum PFLC concentration of 0.75-0.9 g/L and heavy chain plasmid concentration of 2.3-2.8 mg/L.

The data shown in this example demonstrates that the titer of anti-BCMA antibody increased in a dose-response relationship based on the concentration of HC expression plasmid and PFLC added to the reaction.

### Example 5

This example illustrates a method for producing an anti-folate receptor alpha (FOLR1) antibody using a heavy chain expressed in the cell free synthesis reaction and a prefabricated light chain (PFLC).

Methods:

The reaction was performed in a Micro 24 bioreactor (Micro-24 MicroReactor System from PALL® Corp.) (xCF190626 IU) comprising a 4 ml batch reaction and standard cell free feed (25% of the initial batch volume added). Dissolved oxygen (DO) and pH were controlled at 20% and 6.95 respectively. At 14 hrs, the DO and pH were adjusted to 80% and 8.0 respectively. The bioreactor temperature was maintained at 25° C.

The extract comprised SBDG381 extract. The heavy chain plasmid reaction concentration was 0.7, 1.1 and 1.5 mg/L. (The two plasmid system HC running concentration was 1.08 mg/L). The PreFab light chain (PFLC) concentration was 0.5, 0.875, and 1.25 g/L. The PhyTip load was 150 microL.

Results

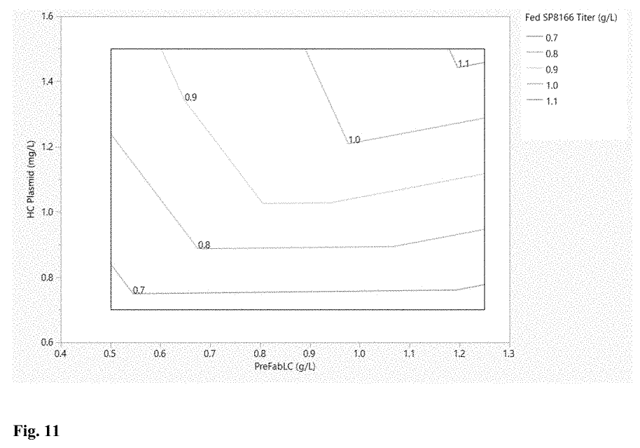

The titer of anti-folate receptor alpha (FOLR1) antibody increased in a dose-response relationship based on the concentration of HC expression plasmid and PFLC added to the reaction. FIG. 11 shows the highest titer of anti-folate receptor alpha (FOLR1) antibody (greater than or equal to 1.1 g/L) was obtained when both PFLC and HC were increased, i.e., to 1.25 g/L and 1.5 mg/L, respectively. A significant titer loss (less than 0.7 g/L) was observed by reducing PFLC and HC at the same time, i.e., to 0.5 g/L and 0.7 mg/L respectively. There was no plasmid system control run for this experiment. Acceptable yields of anti-folate receptor alpha (FOLR1) antibody were obtained with a minimum PFLC concentration of 0.9-1.0 g/L and heavy chain plasmid concentration of 1.5-2.0 mg/L. No further increase in antibody titer was observed when the HC plasmid concentration was increased above 1.5 mg/L (to 2.0 mg/L and 2.5 mg/L) and the PFLC concentration was 1.25 g/L (data not shown).

This data presented in this example shows that the titer of anti-folate receptor alpha (FOLR1) antibody increased in a dose-response relationship based on the concentration of HC expression plasmid and PFLC added to the reaction.

### Example 6

This example illustrates a method for producing an anti-CD74 antibody using a heavy chain expressed in the cell free synthesis reaction and a prefabricated light chain (PFLC).

Methods:

The reaction was performed in a Micro 24 bioreactor (Micro-24 MicroReactor System from PALL® Corp.) (xCF190702 IU) comprising a 4 ml batch reaction and standard cell free feed (25% of the initial batch volume added). Dissolved oxygen (DO) and pH were controlled at 20% and 7.2 respectively. At 14 hrs, the DO and pH were adjusted to 80% and 8.0 respectively. The bioreactor temperature was maintained at 28° C.

The extract comprised SBDG381 extract. The heavy chain plasmid reaction concentration was 3.5, 4.5 and 5.5 mg/L. (The two plasmid system HC running concentration was 4.286 mg/L). The PreFab light chain (PFLC) concentration was 0.6, 0.95, and 1.30 g/L. The PhyTip load was 150 microL, and the plate reader load was 50 microL.

Results

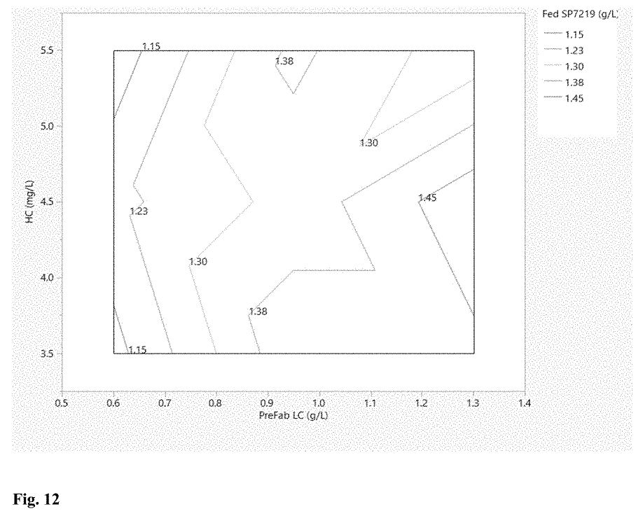

FIG. 12 shows that the highest expression of anti-CD74 antibody is achieved by reducing HC expression plasmid concentrations to 3.5 mg/L and increasing PFLC to 1.3 g/L. Increasing the heaving chain plasmid to concentrations above 4.5 mg/L tended to reduce the antibody titer at higher PFLC concentrations (0.9-1.3 g/L). Acceptable yields of anti-CD74 antibody were obtained with a minimum PFLC concentration of 0.8-0.9 g/L and a HC plasmid concentration of 3.5-3.8 mg/L. No two plasmid control system was included for this experiment.

The above Examples demonstrate that reactions containing an expression plasmid encoding the HC and a pre-fabricated LC that was added to the reactions produced higher titers of antibodies compared to reactions containing expression plasmids encoding both the HC and LC.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

ILLUSTRATIVE SEQUENCES
Protein sequences of IgG HC and LC
*denotes site of NNAA
WT B10 HC
(SEQ ID NO: 1)
MEVQLVESGGGLVQPGGSLRLSCAASGFNTTTKSIHWVRQAPGKGLEWVG
EIYPRDGITDYADSVKGRFTISADTSKNTAYLQMNSLRAEDTAVYYCARG
GWHWRSGYSYYLDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK
WT B10 LC or H01 LC
(SEQ ID NO: 2)
MDIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
B10 F404TAG HC
(SEQ ID NO: 3)
MEVQLVESGGGLVQPGGSLRLSCAASGFNTTTKSIHWVRQAPGKGLEWVG
EIYPRDGITDYADSVKGRFTISADTSKNTAYLQMNSLRAEDTAVYYCARG
GWHWRSGYSYYLDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGS*FLYSKLTVDKSRWQQGNVFSCSVMHEALHNHYTQKSLS
LSPGK
B10 Y180TAG HC
(SEQ ID NO: 4)
MEVQLVESGGGLVQPGGSLRLSCAASGFNTTTKSIHWVRQAPGKGLEWVG
EIYPRDGITDYADSVKGRFTISADTSKNTAYLQMNSLRAEDTAVYYCARG
GWHWRSGYSYYLDYWGQGTLVTVSSASTKGPSVFPLAPSSKSTSGGTAAL
GCLVKDYFPEPVTVSWNSGALTSGVHTFPAVLQSSGL*SLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK
B10 K42TAG LC
(SEQ ID NO: 5)
MDIQMTQSPSSLSASVGDRVTITCRASQDVNTAVAWYQQKPG*APKLLIY
SASFLYSGVPSRFSGSRSGTDFTLTISSLQPEDFATYYCQQHYTTPPTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
B10 Y180TAG F404TAG HC
(SEQ ID NO: 6)
MEVQLVESGGGLVQPGGSLRLSCAASGFNTTTKSIHWVRQAPGKGLEWVG
EIYPRDGITDYADSVKGRFTISADTSKNTAYLQMNSLRAEDTAVYYCARG
GWHWRSGYSYYLDYWGQGTLVTVSSASTKGPSVFPLAPSSKSTSGGTAAL
GCLVKDYFPEPVTVSWNSGALTSGVHTFPAVLQSSGL*SLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGS*FLYSKLTVDKSRWQQGNVFSCSVMHEALHNHYTQKSLS
LSPGK
B10 241TAG HC
(SEQ ID NO: 7)
MEVQLVESGGGLVQPGGSLRLSCAASGFNTTTKSIHWVRQAPGKGLEWVG
EIYPRDGITDYADSVKGRFTISADTSKNTAYLQMNSLRAEDTAVYYCARG
GWHWRSGYSYYLDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSV*L
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK
H01 Y180TAG F404TAG HC
(SEQ ID NO: 8)
MEVQLVESGGGLVQPGGSLRLSCAASGFNIRTQSIHWVRQAPGKGLEWIG
DIFPIDGITDYADSVKGRFTISADTSKNTAYLQMNSLRAEDTAVYYCARG
SWSWPSGMDYYLDYWGQGTLVTVSSASTKGPSVFPLAPSSKSTSGGTAAL
GCLVKDYFPEPVTVSWNSGALTSGVHTFPAVLQSSGL*SLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGS*FLYSKLTVDKSRWQQGNVFSCSVMHEALHNHYTQKSLS
LSPGK
7219 LC
(SEQ ID NO: 9)
MDIQMTQSPSSVSASVGDRVTITCRASQGIGSWLAWYQQKPGKAPKLLIY
AADRLQSGVPSRFSGSGSGTDFTLTISSLQPEDFATYYCQQYHTYPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
7219 F404TAG HC
(SEQ ID NO: 10)
MQVQLVESGGGVVQPGRSLRLSCAASGFNFSDYGMHWVRQAPGKGLEWVA
VIWYDGSISYYADSVKGRFTISRDNSKNTLYLQMNSLRAEDTAVYYCARG
GTVEHGAVYGTDVWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGS*FLYSKLTVDKSRWQQGNVFSCSVMHEALHNHYTQKSLSL
SPGK
αPD1 HC
(SEQ ID NO: 11)
MEVQLVQSGAEVKKPGASVKVSCKASGYTFDSYGISWVRQAPGQGLEWMG
WISAYNGNTNYAQKLQGRVTMTTDTSTNTAYMELRSLRSDDTAVYYCARD
VDYGTGSGYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCEVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
αPD1 LC
(SEQ ID NO: 12)
MSYELTQPPSVSVSPGQTARITCSGDALPKQYAYWYQQKPGQAPVMVIYK
DTERPSGIPERFSGSSSGTKVTLTISGVQAEDEADYYCQSADNSITYRVF
GGGTKVTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS
αTim3 HC
(SEQ ID NO: 13)
MEVQLVESGGGLVQPGGSLRLSCAASGFNIDRYYIHWVRQAPGKGLEWVA
GITPVRGYTEYADSVKDRFTISADTSKNTAYLQMNSLRAEDTAVYYCARG
YVYRMWDSYDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCEVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
αTim3 LC
(SEQ ID NO: 14)
MDIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
αLAG3 HC
(SEQ ID NO: 15)
MQVQLVESGGGVVQPGRSLRLSCAASGFTFSSYGMHWVRQAPGKGLEWVA
VIWYDGSYKYYADSVKGRFTISRDNSKNTLYLQMNSLRAEDTAVYYCARE
EAPENWDYALDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCEVVDVSHEDPEVKFNWYVBGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK
αLAG3 LC
(SEQ ID NO: 16)
MEIVLTQSPGTLSLSPGERATLSCRASQSVSSSYLAWYQQKPGQAPRLLI
YGASSRATGIPDRFSGSGSGTDFTLTISRLEPEDFAVYYCQQYGRSPFSF
GPGTKVDIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC
H01 HC
(SEQ ID NO: 17)
MEVQLVESGGGLVQPGGSLRLSCAASGFNIRTQSIHWVRQAPGKGLEWIG
DIFPIDGITDYADSVKGRFTISADTSKNTAYLQMNSLRAEDTAVYYCARG
SWSWPSGMDYYLDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK
7219 HC
(SEQ ID NO: 18)
MQVQLVESGGGVVQPGRSLRLSCAASGFNFSDYGMHWVRQAPGKGLEWVA
VIWYDGSISYYADSVKGRFTISRDNSKNTLYLQMNSLRAEDTAVYYCARG
GTVEHGAVYGTDVWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPGK
T7 promoter
(SEQ ID NO: 19)
TAATACGACTCACTATAGGG
Trastuzumab HC
(SEQ ID NO: 20)
MEVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSRW
GGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
Ribosomal binding sequence for heavy
chain (SEQ ID NO: 21):
AX₁GAGX₂T,
(wherein X₁ is A or G and X₂ is A or G)
Ribosomal binding sequence for light
chain (SEQ ID NO: 22):
AX₁X₂AX₃AT
(wherein X₁ is G or A, X₂ is G or A, and
X₃ is G or T)

1. A method for large scale production of an antibody using a cell-free protein synthesis system, comprising expressing a heavy chain (HC) polypeptide of an antibody from a nucleic acid encoding the heavy chain in the presence of a light chain (LC) polypeptide, thereby producing the antibody, wherein said expressing is performed in a reaction mixture having a volume of about 10 to about 25,000 liters.

2. The method of claim 1, wherein the reaction mixture comprises a volume of about 10 to about 10,000 liters.

3. (canceled)

4. The method of claim 1, wherein the reaction mixture comprises a volume of about 10,000 liters to about 20,000 liters.

5. The method of claim 1, wherein the reaction mixture comprises a bacterial extract, and the expressing comprises:
(i) combining the bacterial extract with a nucleic acid encoding the HC; and
(ii) incubating the cell-free protein synthesis system under conditions permitting the expression of the HC.

6. The method of claim 1, wherein the yield per liter of total antibody protein and properly folded antibody is increased compared to a reaction mixture where both the heavy and light chain polypeptides are expressed in the same reaction mixture.

7. The method of claim 1, wherein the yield per liter of properly folded antibody is about 30% higher or greater compared to a reaction mixture where both the heavy and light chain polypeptides are expressed in the same reaction mixture.

8. (canceled)

9. The method of claim 1, wherein the yield per liter of properly folded antibody is about 50% higher or greater compared to a reaction mixture where both the heavy and light chain polypeptides are expressed in the same reaction mixture.

10. (canceled)

11. (canceled)

12. The method of claim 1, wherein dimerization between the heavy and light chain polypeptides is increased compared to a reaction mixture where both the heavy and light chain polypeptides are expressed in the same reaction mixture.

13. The method of claim 1, wherein the ratio of heavy and light chain polypeptide dimers to heavy and light chain polypeptide monomers is increased compared to a reaction mixture where both the heavy and light chain polypeptides are expressed in the same reaction mixture.

14. The method of claim 1, wherein the light chain polypeptide is added to the reaction mixture prior to the expressing step.

15. The method of claim 14, wherein the light chain polypeptide is produced in a separate reaction, synthesized or prefabricated before being added to the reaction mixture.

16. The method of claim 14, wherein the light chain polypeptide is expressed from a nucleic acid encoding the light chain polypeptide in a cell-free protein synthesis system, reaction mixture, or cell free extract.

17. The method of claim 14, wherein the light chain polypeptide is expressed from a nucleic acid encoding the LC polypeptide in an intact living cell selected from a bacterial cell or mammalian cell.

18. (canceled)

19. The method of claim 14, wherein the light chain polypeptide is purified or partially purified from cell-free protein synthesis systems, reaction mixtures, cell free extracts, or from cultures of cells before being added to the reaction mixture.

20. The method of claim 1, wherein the antibody is an IgG, IgA, or IgD subtype, or a combination thereof.

21. The method of claim 1, wherein the antibody is a monoclonal antibody.

22. (canceled)

23. The method of claim 1, wherein the antibody comprises a FAB fragment or the antibody is a bispecific antibody.

24. (canceled)

25. The method of claim 23, wherein the bispecific antibody comprises a heterodimeric Fc region comprising two asymmetric CH3 domains that include sequences from IgA and IgG CH3 domains, the bispecific antibody is a domain-exchanged antibody, wherein the HC dimerizes with the LC, the bispecific antibody comprises engineered CH3 domains with enhanced HC heterodimerization based on steric or electrostatic complementarity, or the bispecific antibody comprises one Fab domain and one scFv domain, where the Fab and scFv domains bind to different antigens.

26. (canceled)

27. (canceled)

28. The method of claim 25, wherein the engineered CH3 domains comprise knob and hole mutations that promote the formation of stable CH3 heterodimers.

29. (canceled)

30. The method of claim 1, wherein the HC and/or the LC comprises at least one non-natural amino acid (nnAA) and the nnAA in the HC is the same or different from the nnAA in the LC.

31. (canceled)

32. (canceled)

33. The method of claim 30, wherein the nnAA is p-acetyl-phenylalanine or p-azidomethyl-L-phenylalanine.

34. The method of claim 1, wherein the method further comprises assembling the HC and LC under non-reducing conditions to produce the antibody.

35. The method of claim 1, wherein the cell free protein synthesis system comprises a bacterial extract with associated co-factors, a bacterial extract prepared from an E. coli strain, an oxidative phosphorylation reaction producing ATP, a reconstituted ribosome system, an exogenous protein chaperone, or a mutant Releasing Factor 1 protein (RF1).

36. (canceled)

37. (canceled)

38. (canceled)

39. (canceled)

40. The method of claim 35, wherein the exogenous protein chaperone is selected from the group consisting of a protein disulfide isomerase (PDI), a peptidyl prolyl cis-trans isomerase (PPI), or a deaggregase.

41. The method of claim 40, wherein the PDI is selected from DsbA, DsbC or DsbG; the PPI is selected from FkpA, SlyD, tig, SurA, or Cpr6; and the deaggregase is selected from IbpA, IbpB, or Skp.

42. (canceled)

43. A cell-free protein synthesis system comprising:
(i) a reaction mixture comprising a bacterial cell extract;
(ii) a nucleic acid encoding a heavy chain polypeptide; and
(iii) a light chain polypeptide;
wherein the reaction mixture has a volume of about 10 to about 25,000 liters.

44. The cell-free protein synthesis system of claim 43, wherein the light chain polypeptide is
(i) produced in a separate reaction, synthesized or prefabricated before being added to the reaction mixture;
(ii) expressed from a nucleic acid encoding the light chain polypeptide in a cell-free protein synthesis system, reaction mixture, or cell free extract; or
(iii) expressed from a nucleic acid encoding the LC polypeptide in an intact living cell, wherein the intact living cell is a bacterial cell or mammalian cell.

45. (canceled)

46. (canceled)

47. (canceled)

48. The cell-free protein synthesis system of claim 43, wherein the light chain polypeptide is purified or partially purified from cell-free protein synthesis systems, reaction mixtures, cell free extracts, or from cultures of cells before being added to the reaction mixture.

49. The cell-free protein synthesis system of claim 43, wherein the reaction mixture comprises ribosomes, ATP, amino acids, and tRNAs, the bacterial cell extract is prepared from an E. coli strain, or the cell-free protein synthesis system further comprises a mutant Releasing Factor 1 protein (RF1).

50. (canceled)

51. The cell-free protein synthesis system of claim 43, further comprising an exogenous protein chaperone.

52. The cell-free protein synthesis system of claim 51, wherein the exogenous protein chaperone is selected from the group consisting of a protein disulfide isomerase (PDI), a peptidyl prolyl cis-trans isomerase (PPI), or a deaggregase.

53. The cell-free protein synthesis system of claim 52, wherein the PDI is selected from DsbA, DsbC or DsbG; the PPI is selected from FkpA, SlyD, tig, SurA, or Cpr6; and the deaggregase is selected from IbpA, IbpB, or Skp.

54. (canceled)
